(19) United States
(12) Patent Application Publication
(10) Pub. No.: US 2023/0403460 A1
(43) Pub. Date: Dec. 14, 2023

(54) TECHNIQUES FOR USING SENSOR DATA TO MONITOR IMAGE-CAPTURE TRIGGER CONDITIONS FOR DETERMINING WHEN TO CAPTURE IMAGES USING AN IMAGING DEVICE OF A HEAD- WEARABLE DEVICE, AND WEARABLE DEVICES AND SYSTEMS FOR PERFORMING THOSE TECHNIQUES

(71) Applicant: Meta Platforms Technologies, LLC, Menlo Park, CA (US)

(72) Inventors: Benjamin Neal Bethurum, Shoreline, WA (US); Willy Huang, San Francisco, CA (US); Hind Hobeika, San Francisco, CA (US); David Sven Woodland, Issaquah, WA (US); Szeyin Lee, Seattle, WA (US); Serge Berig, Brooklyn, NY (US)

(21) Appl. No.: 18/330,288

(22) Filed: Jun. 6, 2023

Related U.S. Application Data
(60) Provisional application No. 63/350,831, filed on Jun. 9, 2022.

(51) Int. Cl. H04N 23/60; G06F 1/16; A61B 5/00; G06V 10/25

(52) U.S. Cl. CPC H04N 23/64; G06F 1/163; A61B 5/681; G06V 10/25

(57) ABSTRACT

Systems and methods are provided for using sensor data from a wrist-wearable device to monitor image-capture trigger conditions for determining when to capture images using an imaging device of a head-wearable device. One example method includes receiving, from a wrist-wearable device communicatively coupled to a head-wearable device, sensor data; and determining, based on the sensor data received from the wrist-wearable device, whether an image-capture trigger condition for the head-wearable device is satisfied. The method further includes in accordance with a determination that the image-capture trigger condition for the head-wearable device is satisfied, instructing an imaging device of the head-wearable device to capture image data.

### CROSS-REFERENCE TO RELATED APPLICATIONS

This application claims priority to U.S. Prov. App. No. 63/350,831, filed on Jun. 9, 2022, and entitled “Techniques For Using Sensor Data To Monitor Image-Capture Trigger Conditions For Determining When To Capture Images Using An Imaging Device Of A Head-Wearable Device, And Wearable Devices And Systems For Performing Those Techniques,” which is incorporated herein by reference.

### TECHNICAL FIELD

The present disclosure relates generally to wearable devices and methods for enabling quick and efficient capture of camera data (e.g., still images and videos) and/or the presentation of a representation of the camera data at a coupled display, more particularly, to wearable devices configured to monitor and detect the satisfaction of image-capture trigger conditions based on sensor data and cause the capture of camera data (e.g., which can be done based solely on an automated determination that the trigger condition is satisfied and without an instruction from the user to capture an image), the transfer of the camera data, and/or the display of a representation of the camera data at a wrist-wearable device.

### BACKGROUND

Users performing physical activities conventionally carry a number of electronic devices to assist them in performing a physical activity. For example, users can carry fitness trackers, smartphones, or other devices that include biometric sensors that track the users' performance during a workout. To take a picture during a workout, a user is normally required to pause, end, or temporarily interrupt their workout to capture the image. Additionally, conventional wearable devices that include a display require a user to bring up their device and/or physically interact with the wearable device to capture or review an image, which takes away from the user's experience and can lead to accidental damage caused to such devices after such devices are dropped or otherwise mishandled due to the difficulties of interacting with such devices while exercising. Further, because conventional wearable devices require user interaction to cause capturing of images during exercise, a user is unable to conveniently access, view, and send a captured image.

As such, there is a need for a wearable device that captures an image without distracting the user or requiring user interaction, especially while the user engages in an exercise activity.

### SUMMARY

To avoid one or more of the drawbacks or challenges discussed above, a wrist-wearable device and/or a head-wearable device monitor respective sensor data from communicatively coupled sensors to determine whether one or more image-capture trigger conditions are satisfied. When the wrist-wearable device and/or a head-wearable device determine that an image-capture trigger condition is satisfied, the wrist-wearable device and/or a head-wearable device cause a communicatively coupled imaging device to automatically capture image data. By automatically capturing image data when an image-capture trigger condition is satisfied (and, e.g., doing so without an express instruction from the user to capture an image such that the satisfaction of the image-capture trigger condition is what causes the image to be captured and not a specific user request or gesture interaction), the wrist-wearable device and/or a head-wearable device reduce the number of inputs required by a user to capture images, as well as reduce the amount of physical interactions that a user needs have with an electronic device, which in turn improve users' daily activities and productivity and help to avoid users damaging their devices by attempting to capture images during an exercise activity. Some examples also allow for capturing images from multiple cameras after an image-capture trigger condition is satisfied, e.g., respective cameras of a head-wearable device and a wrist-wearable device both capture images, and those multiple images can be shared together and can also be overlaid with exercise data (e.g., elapsed time for a run, average pace, etc.).

The wrist-wearable devices, head-wearable devices, and methods described herein, in one embodiment, provide improved techniques for quickly capturing images and sharing them with contacts. In particular, a user wearing a wrist-wearable device and/or head-wearable devices, in some embodiments, can capture images as they travel, exercise, and/or otherwise participate in real-world activities. The non-intrusive capture of images do not exhaust power and processing resources of a wrist-wearable device and/or head-wearable device, thereby extending the battery life of each device. Additional examples are explained in further detail below.

### BRIEF DESCRIPTION OF THE DRAWINGS

So that the present disclosure can be understood in greater detail, a more particular description may be had by reference to the features of various embodiments, some of which are illustrated in the appended drawings. The appended drawings, however, merely illustrate pertinent features of the present disclosure. The description may admit to other effective features as the person of skill in this art will appreciate upon reading this disclosure.

FIGS. 1A-1B-3 illustrate the automatic capture of image data, in accordance with some embodiments.

FIGS. 1C and 1D illustrate the transfer of image data and the presentation of image data between different devices, in accordance with some embodiments.

FIGS. 1E-1F-5 illustrate the presentation and editing of a representation of the image data and the selection of different image data, in accordance with some embodiments.

FIGS. 1G-1J illustrate different user interfaces for sharing the captured image data with other users, in accordance with some embodiments.

FIGS. 1K-1L illustrate automatically sharing the captured image data, in accordance some embodiments.

FIGS. 1M-1N illustrate one or more messages received and presented to the user during a physical activity, in accordance with some embodiments.

FIGS. 1O-1P illustrate one or more responses that the user can provide to received messages during a physical activity, in accordance with some embodiments.

FIG. 2 illustrates a flow diagram of a method for using sensor data from a wrist-wearable device to monitor image-capture trigger conditions for determining when to capture images using an imaging device of a head-wearable device, in accordance with some embodiments.

FIG. 3 illustrates a detailed flow diagram of a method of using sensor data from a wrist-wearable device to monitor image-capture trigger conditions trigger conditions for determining when to capture images using an imaging device of a head-wearable device, in accordance with some embodiments

FIGS. 4A-4F illustrate using sensor data from a wrist-wearable device to perform one or more operations via a communicatively coupled head-wearable device, in accordance with some embodiments.

FIG. 5 is a detailed flow diagram illustrating a method for unlocking access to a physical item using a combination of a wrist-wearable device and a head-wearable device.

FIGS. 6A-6E illustrate an example wrist-wearable device, in accordance with some embodiments.

FIGS. 7A-7B illustrate an example AR system in accordance with some embodiments.

FIGS. 8A and 8B are block diagrams illustrating an example artificial-reality system in accordance with some embodiments.

In accordance with common practice, like reference numerals may be used to denote like features throughout the specification and figures.

### DETAILED DESCRIPTION

Numerous details are described herein to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not necessarily been described in exhaustive detail so as to avoid obscuring pertinent aspects of the embodiments described herein.

Embodiments of this disclosure can include or be implemented in conjunction with various types or embodiments of artificial-reality systems. Artificial-reality (AR), as described herein, is any superimposed functionality and or sensory-detectable presentation provided by an artificial-reality system within a user's physical surroundings. Such artificial-realities can include and/or represent virtual reality (VR), augmented reality, mixed artificial-reality (MAR), or some combination and/or variation one of these. For example, a user can perform a swiping in-air hand gesture to cause a song to be skipped by a song-providing API providing playback at, for example, a home speaker. An AR environment, as described herein, includes, but is not limited to, VR environments (including non-immersive, semi-immersive, and fully immersive VR environments); augmented-reality environments (including marker-based augmented-reality environments, markerless augmented-reality environments, location-based augmented-reality environments, and projection-based augmented-reality environments); hybrid reality; and other types of mixed-reality environments.

Artificial-reality content can include completely generated content or generated content combined with captured (e.g., real-world) content. The artificial-reality content can include video, audio, haptic events, or some combination thereof, any of which can be presented in a single channel or in multiple channels (such as stereo video that produces a three-dimensional effect to a viewer). Additionally, in some embodiments, artificial reality can also be associated with applications, products, accessories, services, or some combination thereof, which are used, for example, to create content in an artificial reality and/or are otherwise used in (e.g., to perform activities in) an artificial reality.

A hand gesture, as described herein, can include an in-air gesture, a surface-contact gesture, and or other gestures that can be detected and determined based on movements of a single hand or a combination of the user's hands. In-air means, in some embodiments, that the user hand does not contact a surface, object, or portion of an electronic device (e.g., the head-wearable device 110 or other communicatively coupled device, such as the wrist-wearable device 120), in other words the gesture is performed in open air in 3D space and without contacting a surface, an object, or an electronic device. Surface-contact gestures (contacts at a surface, object, body part of the user, or electronic device) more generally are also contemplated in which a contact (or an intention to contact) is detected at a surface (e.g., a single or double finger tap on a table, on a user's hand or another finger, on the user's leg, a couch, a steering wheel, etc.). The different hand gestures disclosed herein can be detected using image data and/or sensor data (e.g., neuromuscular signals sensed by one or more biopotential sensors (e.g., EMG sensors) or other types of data from other sensors, such as proximity sensors, time-of-flight sensors, sensors of an inertial measurement unit, etc.) detected by a wearable device worn by the user and/or other electronic devices in the user's possession (e.g., smartphones, laptops, imaging devices, intermediary devices, and/or other devices described herein).

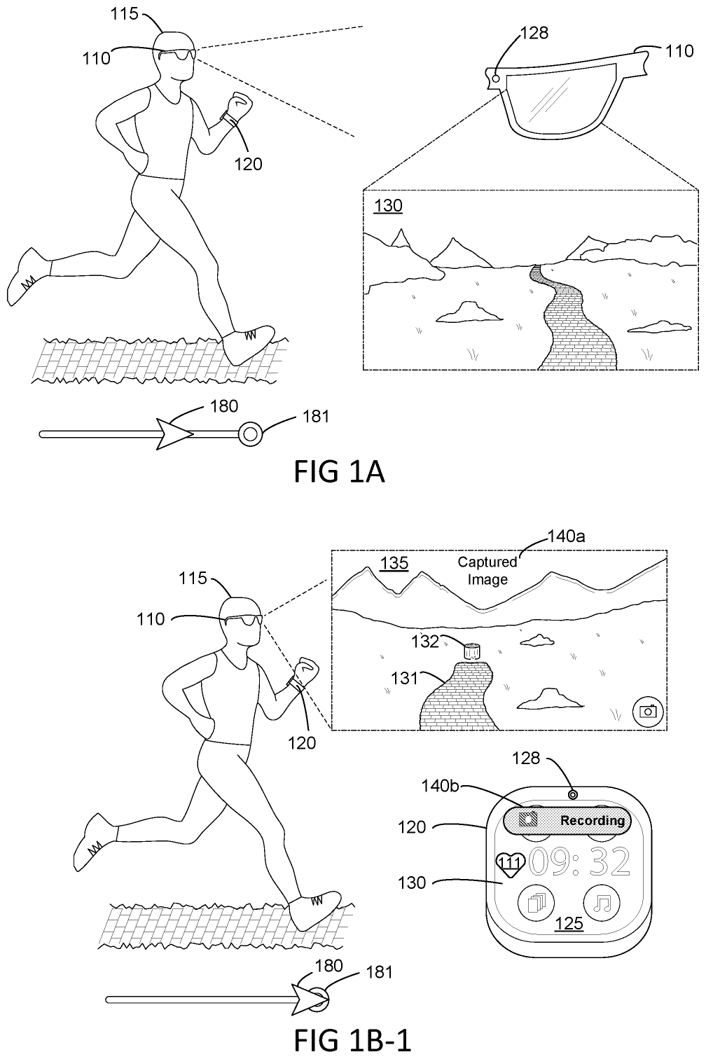

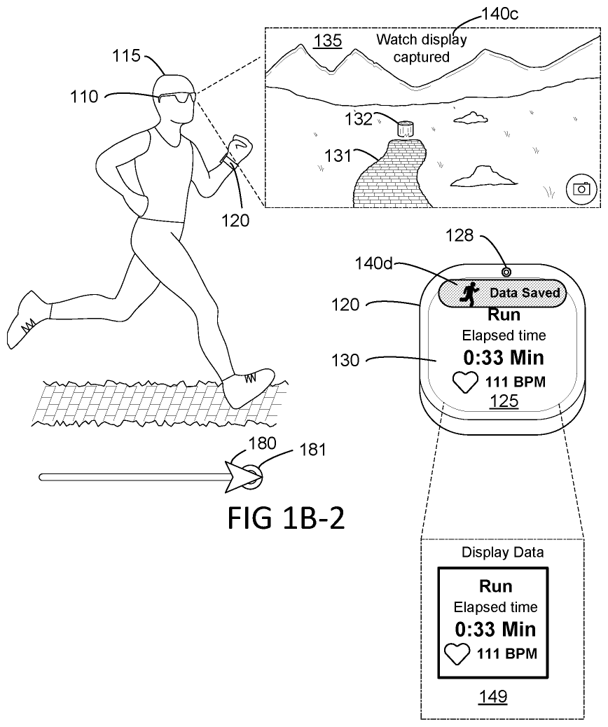

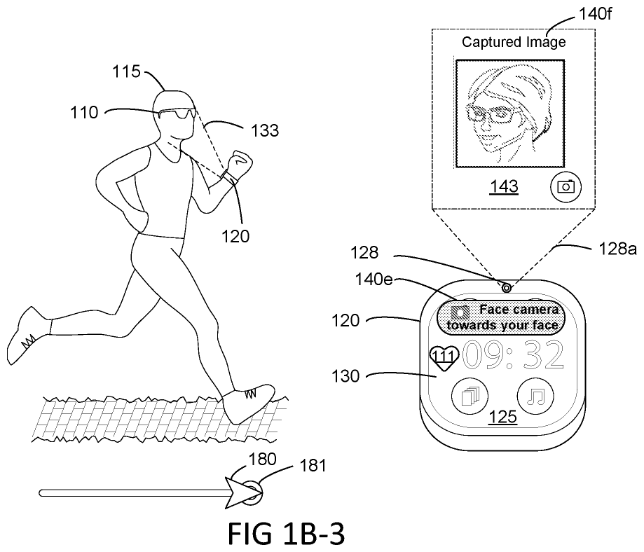

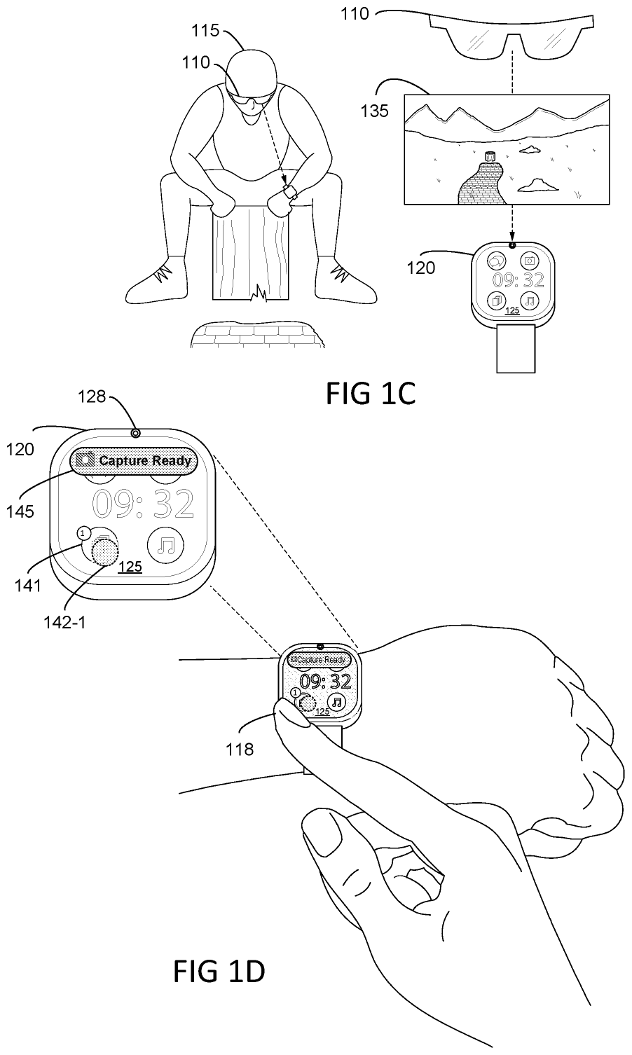

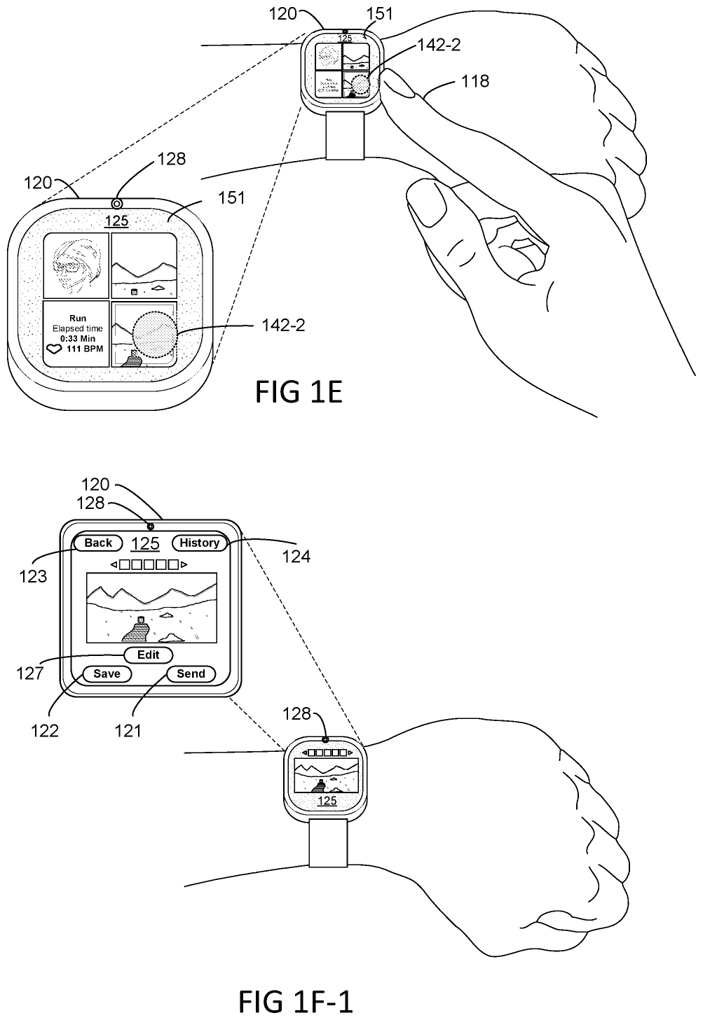

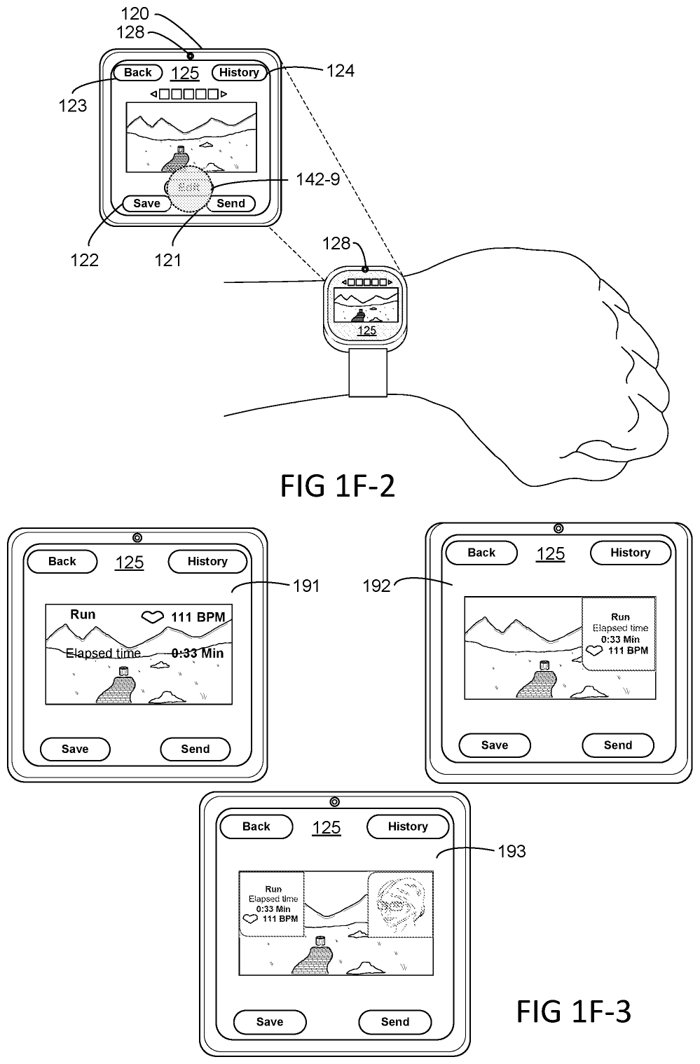

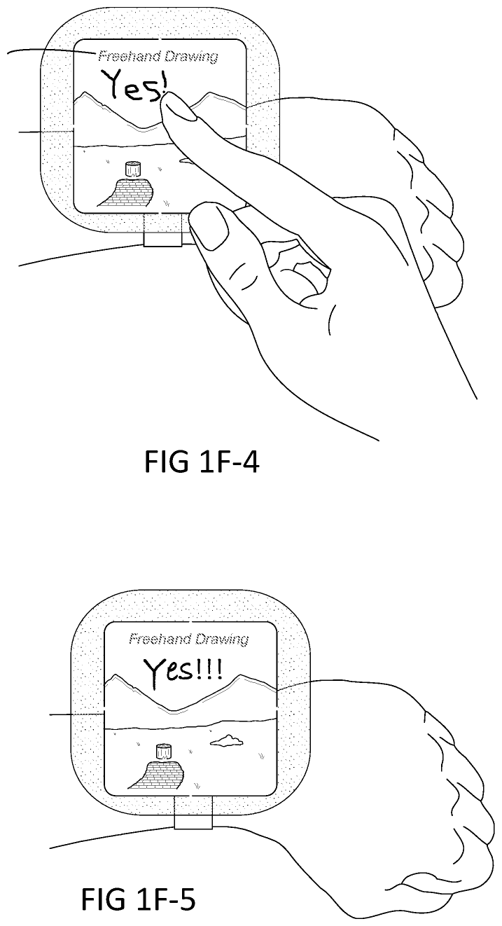

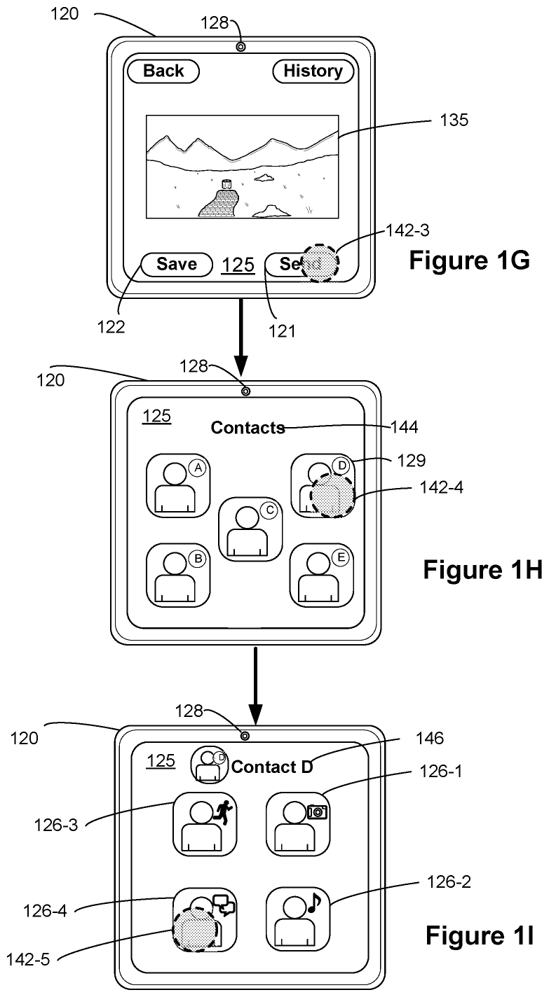

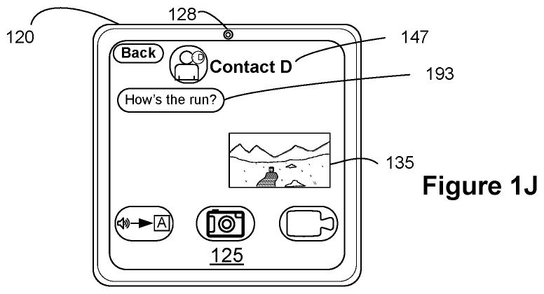

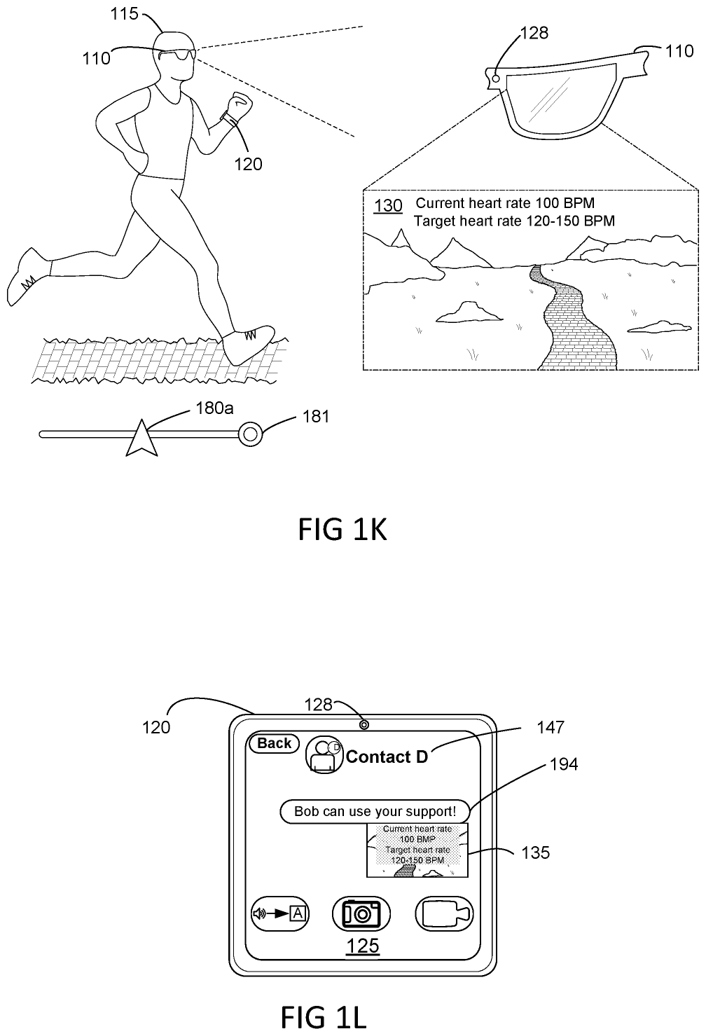

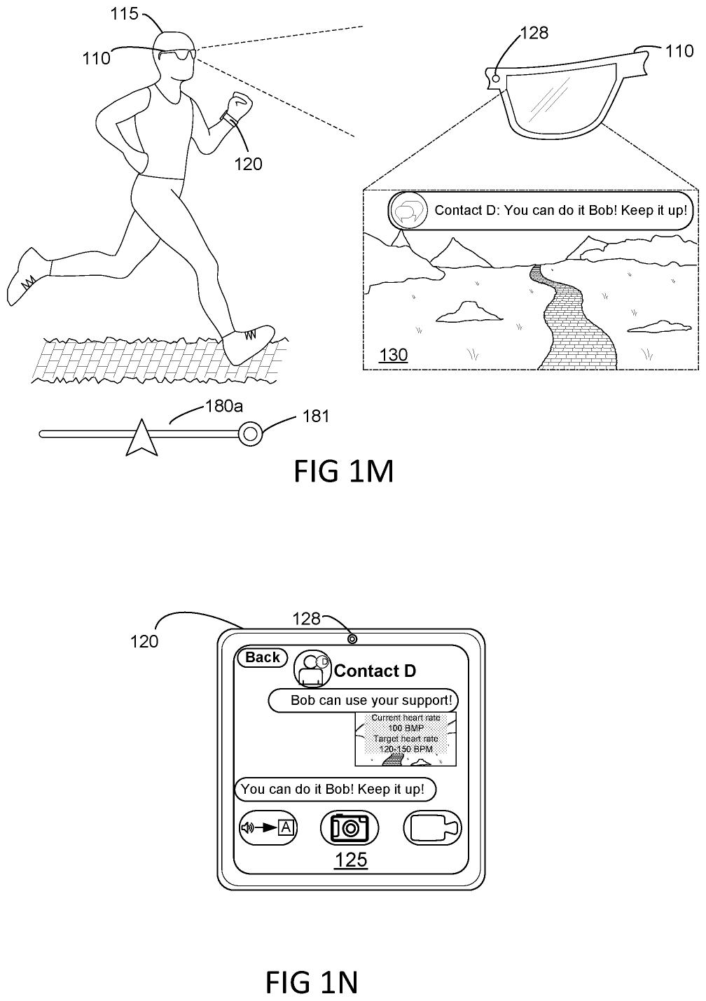

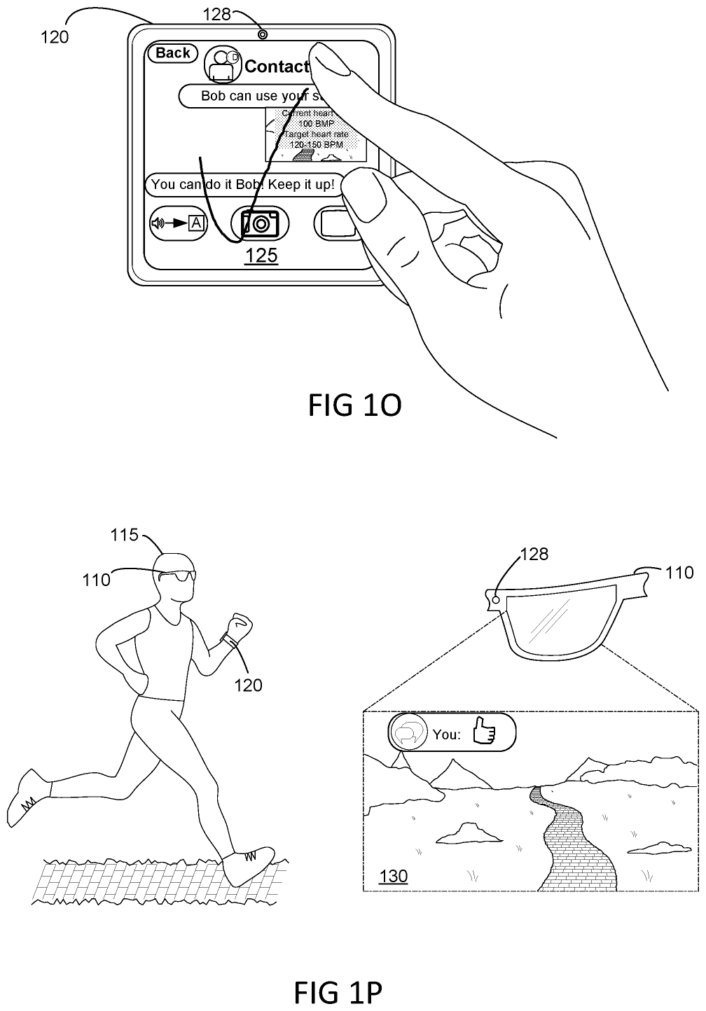

FIGS. 1A-1I illustrate using sensor data from a wrist-wearable device to monitor trigger conditions for determining when to capture images using an imaging device of a head-wearable device, in accordance with some embodiments. In particular, the user 115 is able to use sensor data of a worn wrist-wearable device 120 and/or head-wearable device 110 to automatically capture image data without having to physically contact the wrist-wearable device 120 and/or a head-wearable device 110. By using the wrist-wearable device 120 and/or head-wearable device 110, the user 115 is able to conveniently capture image data 135 and reduce the amount of time required to capture image data by reducing the overall of inputs and/or the physical interaction required by the user 115 at an electronic device coupled with an imaging device 128 for capturing the image data. Thus, the user 115 can focus on real-world activities (e.g., exercise) and need not keep gesturing to capture images, instead they can configure image-capture trigger condition beforehand and know that the system will capture images at appropriate times without needed any specific requests to cause the image captures each time.

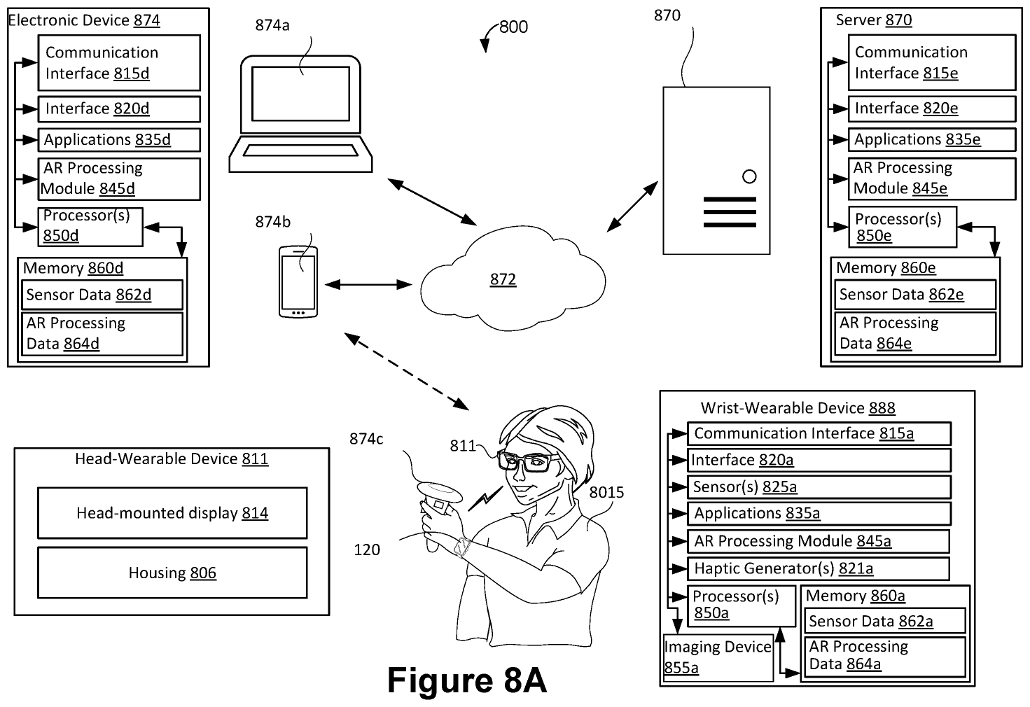

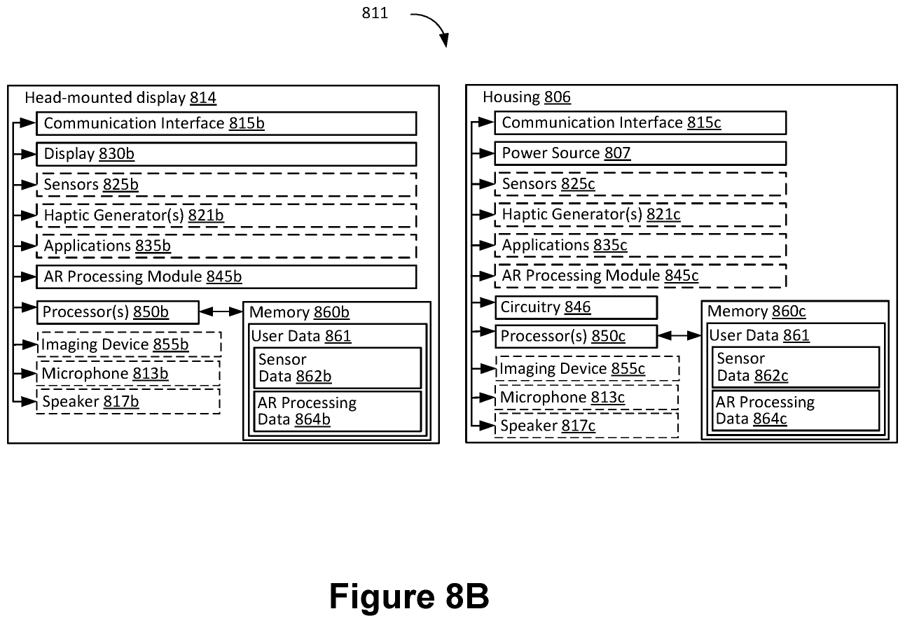

The wrist-wearable device 120 can include includes one or more displays 130 (e.g., a touch screen 125) for presenting a visual representation of data to a user 115, speakers for presenting an audio representation of data to the user 115, microphones for capturing audio data, imaging devices 128 (e.g., a camera) for capturing image data and/or video data (referred to as “camera data”), and sensors (e.g., sensors 825, such as electromyography (EMG) sensors, inertial measurement units (IMU)s, biometric sensors, position sensors, and/or any other sensors described below in reference to FIGS. 8A-8B) for detecting and determining satisfaction of one or more image-capture trigger conditions. In some embodiments, the one or more components of the wrist-wearable device 120 described above are coupled with a wrist-wearable structure (e.g., a band portion) of the wrist-wearable device 120, housed within a capsule portion of the wrist-wearable device 120 or a combination of the wrist-wearable structure and the capsule portion.

The head-wearable device 110 includes one or more imaging devices 128, microphones, speakers, displays 130 (e.g., a heads-up display, a built-in or integrated monitor or screen, a projector, and/or similar device), and/or sensors. In some embodiments, the head-wearable device 110 is configured to capture audio data via an microphone and/or present a representation of the audio data via speakers. In some embodiments, the head-wearable device 110 is a pair of smart glasses, augmented reality goggles (with or without a heads-up display), augmented reality glasses (with or without a heads-up display), other head-mounted displays, or head-wearable device 110). In some embodiments, the one or more components of the head-wearable device 110 described above are coupled with the housing and/or lenses of the head-wearable device 110. The head-wearable device can be used in real-world environments and/or in AR environments. For example, the head-wearable device can capture image data while a user walks, cooks, drives, jogs, or performs another physical activity without requiring user interaction at the head-wearable device or other device communicatively coupled with the head-wearable device.

In some embodiments, the wrist-wearable device 120 can communicatively couple with the head-wearable device 110 (e.g., by way of a Bluetooth connection between the two devices, and/or the two devices can also both be connected to an intermediary device such as a smartphone 874a that provides instructions and data to and between the two devices). In some embodiments, the wrist-wearable device 120 and the head-wearable device 110 are communicatively coupled via an intermediary device (e.g., a server 870, a computer 874a, a smartphone 874b and/or other devices described below in reference to FIGS. 8A-8B) that is configured to control the wrist-wearable device 120 and head-wearable device 110 and/or perform one or more operations in conjunction the operations performed by the wrist-wearable device 120 and/or head-wearable device 110.

The wrist-wearable device 120 and/or the head-wearable device 110 worn by the user 115 can monitor, using data obtained by one or more communicatively coupled sensors, user movements (e.g., arm movements, wrist movements, head movements, and torso movements), physical activity (e.g., exercise, sleep), location, biometric data (e.g., hear rate, body temperature, oxygen saturation), etc. The data obtained by the one or more communicatively coupled sensors can be used by the wrist-wearable device 120 and/or the head-wearable device 110 to capture image data 135 (e.g., still images, video, etc.) and/or share the image data 135 to other devices, as described below.

In some embodiments, the wrist-wearable device 120 is configured to instruct a communicatively coupled imaging device 128 (e.g., imaging device 128 of the head-wearable device 110) to capture image data 135 when the sensor data, sensed by the wrist-wearable device 120 (or other communicatively coupled device), satisfies an image-capture trigger condition. The instruction to capture image data 135 can be provided shortly after a determination that the sensor data satisfies an image-capture trigger condition (e.g., within 2 ms of the determination). Further, the instruction to capture image data 135 can be provided without any further user instruction to capture the image (e.g., the system (e.g., the communicatively coupled wrist-wearable device 120 and head-wearable device 110) proceeds to capture the image data 135 because the image-capture trigger condition was satisfied and does not need to receive any specific user request beforehand). For example, wrist-wearable device 120 can provide instructions to the head-wearable device 110 that cause the imaging device 128 of the head-wearable device 110 to capture image data of the user 115's field of view (as described below in reference to FIGS. 1B-1-1B-3).

The image-capture trigger conditions can include biometric triggers (e.g., heart rate, SPO2, skin conductance), location triggers (e.g., a landmark, a particular distance, a percentage of a completed route, a user-defined location, etc.), user position triggers (e.g., head position, distance traveled), computer vision based trigger (e.g., objects detected in the image data), movement triggers (e.g., user velocity, user pace), physical activity triggers (e.g., elapsed workout times, personal record achievements), etc. The image-capture trigger conditions can be user-defined and/or predefined. For example, the user 115 can set a target heart rate to be an image-capture trigger condition, such that when the user 115's heart rate reaches the target the image-capture trigger condition is satisfied. In some embodiments, one or more image-capture trigger conditions are generated and updated over predetermined period of time (e.g., based on the user 115's activity or history). For example, the image-capture trigger condition be a running pace that is determined based on the user 115's previous workouts over a predetermined period of time (e.g., 5 day, two weeks, a month).

The wrist-wearable device 120 can determine whether one or more image-capture trigger conditions are satisfied based on sensor data from at least one sensor. For example, the wrist-wearable device 120 can use the user 115's hear rate to determine that an image-capture trigger condition is satisfied. Alternatively or in addition, in some embodiments, the wrist-wearable device 120 can determine that one or more image-capture trigger conditions are satisfied based on a combination of sensor data from at least two sensors. For example, the wrist-wearable device 120 can use a combination of the user 115's heart rate and the user 115's running pace to determine that another image-capture trigger condition is satisfied. The above examples are non-limiting; the sensor data can include biometric data (e.g., heart rate, O2), performance metrics (e.g., elapsed time, distance), position data (e.g., GPS, location), image data 135 (e.g. identified objects, such as landmarks, animals, flags, sunset, sunrise), acceleration data (e.g., sensed by one or more accelerometers), EMG sensor data, IMU data, as well as other sensor data described below in reference to FIGS. 8A-8B. Any combination of sensor data received by the wrist-wearable device 120 and/or head-wearable device 110 can be used to determine whether an image-capture trigger condition is satisfied.

In some embodiments, sensor data from one or more sensors of different devices can be used to determine whether an image-capture trigger condition is satisfied. For example, data obtained by one or more sensors of a head-wearable device 110 worn by the user 115 and data obtained by one or more sensors of a wrist-wearable device 120 worn by the user 115 can be used to determine that an image-capture trigger condition is satisfied. In some embodiments, the sensor data is shared between communicatively coupled devices (e.g., both the head-wearable device 110 and the wrist-wearable device 120 have access to the data obtained by their respective sensors) such that each device can determine whether an image-capture trigger condition is satisfied and/or to verify a determination that an image-capture trigger condition is satisfied. Alternatively, in some embodiments, the sensor data is received at a single device, which determines whether an image-capture trigger condition is satisfied. For example, a head-wearable device 110 worn by a user can provide data obtained by its one or more sensors to a wrist-wearable device 120 such that the wrist-wearable device 120 can determine whether an image-capture trigger condition is satisfied (e.g., using sensor data of the wrist-wearable device 120 and/or head-wearable device 110).

Additionally or alternatively, in some embodiments, the wrist-wearable device 120 and/or the head-wearable device 110 can determine whether an image-capture trigger condition is satisfied based, in part, on image data captured by an imaging device 128 communicatively coupled with the wrist-wearable device 120 and/or the head-wearable device 110. For example, the head-wearable device 110 can process image data (before capture) of a field of view a coupled imaging device 128 to identify one or more predefined objects, such as landmarks, destinations, special events, people, animals, etc., and determine whether an image-capture trigger condition is satisfied based on the identified objects. Similarly, the head-wearable device 110 can provide transient image data (e.g., image data that is not permanently stored) of a field of view a coupled imaging device 128 to the wrist-wearable device 120, which in turn processes the transient image data to determine whether an image-capture trigger condition is satisfied based on the identified objects.

Image data 135 captured in response to the instructions provided by the wrist-wearable device 120 (when an image-capture trigger condition is satisfied) can be transferred between the user 115's communicatively coupled devices and/or shared with electronic devices of other users. In some embodiments, the instructions provided by the wrist-wearable device 120 to capture the image data 135 can further cause the presentation of the image data 135 via a communicatively coupled display 130. In particular, the wrist-wearable device 120, in conjunction with instructing a communicatively coupled imaging device 128 to capture image data 135, can provide instructions to cause a representation of the image data 135 to be presented at a communicatively coupled display (e.g., display 130 of the head-wearable device 120) and transferred from imaging device to other devices (e.g., from the imaging device 128 of the head-wearable device 110 to the wrist-wearable device 120). Further, in some embodiments, image-capture trigger conditions can be associated with one or more commands other than capturing image data, such as opening an application, activating a microphone, sending a message, etc. For example, instruction provided by the wrist-wearable device 120 responsive to satisfaction of an image-capture trigger condition, can further causes a microphone of a head-wearable device 110 to be activated such that audio data can be captured in conjunction with image data 135.

While the examples above describe the wrist-wearable device 120 and/or the head-wearable device 110 determining whether an image-capture trigger condition is satisfied, intermediary devices communicatively coupled with the wrist-wearable device 120 and/or the head-wearable device 110 can determine, alone or in conjunction with the wrist-wearable device 120 and/or the head-wearable device 110, whether an image-capture trigger condition is satisfied. For example, the wrist-wearable device 120 and/or the head-wearable device 110 can provide data obtained via one or more sensors to a smartphone 874b, which in turn determines whether an image-capture trigger condition is satisfied.

Turning to FIG. 1A, the user 115 is exercising outdoors while wearing the head-wearable device 110 and the wrist-wearable device 120. While worn by the user 115, the wrist-wearable device 120 and/or the head-wearable device 110 monitor sensor data to determine whether an image-capture trigger condition is satisfied. One or all of the sensors of a wrist-wearable device 120 and/or a head-wearable device 110 can be utilized to provide data for determining that an image-capture trigger is satisfied. For example, while the user 115 wearing the wrist-wearable device 120 and/or the head-wearable device 110 performs a physical activity, the wrist-wearable device 120 and/or the head-wearable device 110 detect the user 115's position data (e.g., current position 180) relative to a distance-based image-capture trigger condition (e.g. target destination 181). The wrist-wearable device 120 and/or the head-wearable device 110, using the one or more processors (e.g., processors 850 FIGS. 8A-8B), determine whether the user 115's current position 180 satisfies the image-capture trigger condition. In FIG. 1A, the wrist-wearable device 120 and/or the head-wearable device 110 determine that the user 115's current position 180 does not satisfy the image-capture trigger condition (e.g., is not at the target destination 181) and forgo providing instructions to coupled imaging device 128 for capturing image data 135. As described above, the image-capture trigger condition (e.g. target destination 181) can be user-defined and/or predetermined based on the user 115's prior workout history, workout goals, fitness level, and/or a number of other factors.

In FIG. 1B-1, the image-trigger capture condition is determined to be satisfied by the one or more processors of the wrist-wearable device 120 and/or the head-wearable device 110. More specifically, the wrist-wearable device 120 and/or the head-wearable device 110 determine that the user 115's current position 180 is at the target destination 181, satisfying the image-capture trigger condition. In accordance with a determination that the image-trigger capture condition is satisfied, the wrist-wearable device 120 and/or the head-wearable device 110 instruct a coupled imaging device 128 to capture image data 135. For example, as shown in FIG. 1B-1, when the user 115 reaches the target destination 181 (which is identified as an image-trigger capture condition), the imaging device 128 of the head-wearable device 110 is instructed to capture image data 135. In some embodiments, after the imaging device 128 captures the image data 135, the head-wearable device 110 and/or the wrist-wearable device present to the user 115, via a coupled display (e.g., the display 130 of the head-wearable device 110), a notification 140a that an image was captured. Similarly, when the imaging device 128 is recording image data 135 (e.g., recording a video) the head-wearable device 110 and/or the wrist-wearable device present to the user 115, via the coupled display (e.g., the display 130 of the wrist-wearable device 120), a notification 140b that the imaging device 128 is recording. The notifications can also include suggestions to the user 115. For example, as described below in reference to FIG. 1B-3, a notification presented to the user 115 can suggest the user 115 to take a selfie using the imaging device 128 on the wrist-wearable device 120, which can be combined or merged with the image data 135 captured by the head-wearable device 110.

As described above, the image-capture trigger conditions can also include one or more predefined objects; such that when a predefined object is detected, the image-capture trigger is satisfied. In some embodiments, a predefined object can be selected based on the user 115's history. For example, if the user 115 has a location he usually rests on his run (i.e., the stump 132 in captured image 135), the user 115 can set or the system can automatically set the resting location (e.g., the stump 132) as an image-capture trigger condition. In an alternate embodiment, the user 115 can set the predefined object to be another person the user 115 might know. For example, if the user 115 sees his friend (which would be in a field of view of the worn head-wearable device 110) while exercising, the imaging device 128 coupled to the head-wearable device 110 can capture image data of the friend. Alternatively or additionally, in some embodiments, the one or more predefined objects can include features of a scene that signify an end point. For example, in FIG. 1B-1, a predefined object can be the end of the path 131 and/or the stump 132 at the end of that path 131, which can be interpreted as an endpoint. The image data 135 sensed by the imaged device 128 of the head-wearable device 110 can be processed (before the image data 135 is captured) to detect presence of a predefined object, and in accordance with a determination a predefined object is present, satisfying an image-capture trigger condition, the wrist-wearable device 120 and/or the head-wearable device 110 instruct the coupled imaging device 128 to capture the image data. For example, in FIG. 1B-1, when the imaging device 128 of the head-wearable device 110 detects the presence of the stump 132 at the end of the path 131, the wrist-wearable device 120 and/or the head-wearable device 110 instruct the coupled imaging device 128 to capture the image data 135.

In an additional embodiment, the image-capture trigger conditions can also include a target heart rate. The wrist-wearable device 120 and/or the head-wearable device 110 can monitor the user 115's heart rate 111, and, when the user 115's heart rate 111 satisfies the target heart rate, the wrist-wearable device 120 and/or the head-wearable device 110 instruct the coupled imaging device 128 to capture the image data 135. The above examples are non-limiting; additional examples of the image-capture triggers are provided above

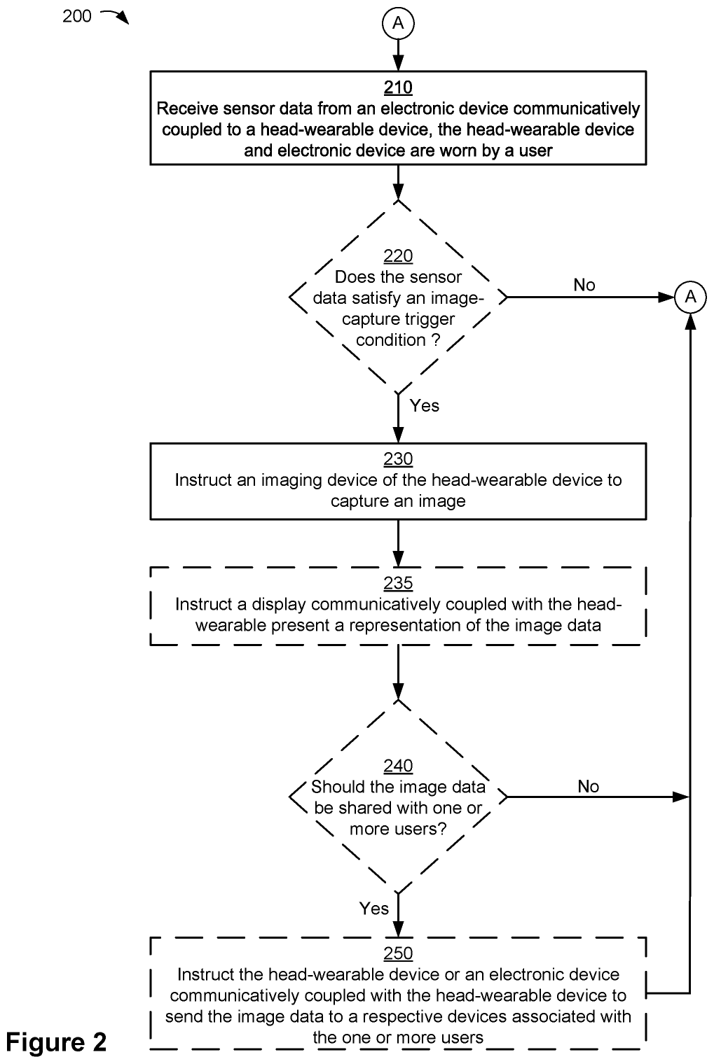

FIG. 1B-2 shows the capture of display data 149 at the wrist-wearable device 120, in accordance with some embodiments. In some embodiments, in accordance with a determination that the image-trigger capture condition is satisfied, the wrist-wearable device 120 is configured capture display data 149 (e.g., a screenshot of the currently displayed information on the display 130). For example, as shown in FIG. 1B-2, when the user 115 reaches the target destination 181, the wrist-wearable device 120 is instructed to capture a screenshot of a fitness application displayed on the display 130 of the wrist-wearable device 120. In some embodiments, after the wrist-wearable device 120 captures the display data 149, the head-wearable device 110 and/or the wrist-wearable device present to the user 115, via a coupled display, a notification 140c and/or 140d that display data 149 was captured. In some embodiments, the notification 140 provides information about the captured display data 149. For example, in FIG. 1B-2 notification 140c notifies the user 115 that the display data 149 was captured from the wrist-wearable device 120 and notification 140d notifies the user that the display data 149 was from a fitness application (represented by the running man icon). Any display 130 communicatively coupled with the wrist-wearable device 120 and/or head-wearable device 110 can be caused to capture display data 149 based on user preference and settings. More specifically, the user 115 can designate one or more devices to capture image data and/or display data 149, as well as restrict one or more devices from capturing image data and/or display data 149.

FIG. 1B-3 illustrates suggestions provided to a user 115 for capturing a selfie image, in accordance with some embodiments. In some embodiments, the head-wearable device 110 and/or the wrist-wearable device 120 provide a notification suggesting the user 115 to position an imaging device 128 of the wrist-wearable device 120 (or other imaging device) such that they are in its field of view 133 of the imaging device 128 for a selfie. For example, as shown in FIG. 1B-3, the display 130 of the wrist-wearable device 120 provides notification 140e suggesting the user 115 to face the camera towards their face. The wrist-wearable device 120 and/or the head-wearable device 110 can provide the user with an additional notification 140f notifying the user that a selfie image 143 was captured.

In FIG. 1C, the user 115 has reached a rest point and paused his workout, which can be detected via the one or more sensors of the wrist-wearable device 120 and/or the head-wearable device 110. In some embodiments, image data 135 can be transferred between the user 115's devices when the user has stopped moving, slowed down their pace, entered a recovery period, reached a rest location, and/or paused the workout. In some embodiments, the user 115 can identify a rest point as an image transfer location such that when the user 115 reaches the transfer location captured image data 135 is automatically transferred between the devices. In some embodiments, the wrist-wearable device 120 and/or the head-wearable device 110 transfer data when the two devices come in close proximity (e.g., within 6 inches) to one another or contact one another. The wrist-wearable device 120 and/or the head-wearable device 110 can transfer image data and/or other data to facilitate the presentation of the transferred data at another device. For example, as shown in FIG. 1C, the image data 135 captured by the imaging device 128 of the head-wearable device 110 is transferred to the wrist-wearable device 120 such that the user 115 can view a representation of the image data 135 from a display of the wrist-wearable device 120.

In some embodiments, the image data 135 is not transferred between devices until the user 115 has stopped moving, reached a rest point, paused their workout, etc. In this way, transfer errors are minimized and the battery of each device is conserved by reducing the overall number of attempts needed to successfully transfer the image data 135. Alternatively or in addition, in some embodiments, the image data 135 is not transferred between the head-wearable device 110 and the wrist-wearable device 120 until the user 115 looks at the wrist-wearable device 120 (initiating the transfer of the captured image 135 from the head-wearable device 110 to the wrist-wearable device 120). In some embodiments, the user 115 can manually initiate a transfer of the captured image 135 from the head-wearable device 110 by inputting one or more commands at the wrist-wearable device 120 (e.g., one or more recognized hand gestures or inputs on a touch screen). In some embodiments, the user 115 can also use voice commands (e.g., “transfer my most recent captured image to my watch”) to transfer the captured image 135 to the wrist-wearable device 120.

In FIG. 1D, the user 115 is notified that the captured image 135 was successfully transferred to wrist-wearable device 120 from the head-wearable device 110. For example, the display 130 of the wrist-wearable device 120 can present a notification 145 that the image data 135 is ready for viewing. In some embodiments, the wrist-wearable device 120 present to the user 115, via display 130, one or more applications, such as a photo gallery icon 141. In some embodiments, user selection 142-1 of the photo gallery icon 141 causes the wrist-wearable device 120 to present a representation of the image data as shown in FIG. 1E. The user 115 can provide an input via a touch screen of the wrist-wearable device 120, a voice command, and/or one or more detected gestures.

FIG. 1E illustrates a photo gallery 151 presented to the user 115 in response to selection of the photo gallery icon 141. In some embodiments, the photo gallery 151 includes one or more representations of the image data 135 captured by the coupled imaging device 128 and/or display data 149 captured by the wrist-wearable device 120 (or other device communicatively coupled with the wrist-wearable device 120 and/or head-wearable device 110). For example, in FIG. 1E, the user's selfie image 143, display data 149, and image data 135 are presented on the display 130 of the wrist-wearable device 120. In some embodiments, a plurality of images is presented to the user 115 via the display 130 of the wrist-wearable device 120. Each representation of the image data 135 and/or display data 149 can be selected by the user 115 to be viewed in detail. The user 115 can select a representation of the image data 135 via user input as described above in reference to FIG. 1D.

In FIG. 1F-1, a representation of the image data 135 selected by the user 115 is presented via display 130 of the wrist-wearable device 120. The representation of the image data 135 is presented in conjunction with one or more selectable affordances that allow the user 115 to save, share and/or edit the representation of the image data 135, display data 149, and/or selfie image 143. In some embodiments, if the user 115 selects the save button 122 the user 115 can save the captured image 135, display data 149, and/or selfie image 143 to one or more applications (e.g., a photo application, a file storage application, etc.) on the wrist-wearable device 120 or other communicatively coupled devices (e.g. a smartphone 874b, a computer 874a, etc.). Additional selectable affordances include a back button 123, which if selected will return to the user 115 to photo gallery 151 described in reference to FIG. 1E. In additional embodiments, a user 115 can select the history button 124 and view information about the captured image 135 such as a time the image data 135, display data 149, and/or selfie image 143 was captured, the device that captured the image data, modifications to the image data, previously captured image data (e.g., at a distinct time), etc. In some embodiments, the user 115 can select the send button 121 which allows the user 115 to share the image data 135, display data 149, and/or selfie image 143 with another user through various methods described below. As described in detail below in reference to FIGS. 1F-2 and 1F-3, in some embodiments, selection of the edit button 127 allows the user 115 to edit the image data 135, display data 149, and/or selfie image 143.

In FIG. 1F-2, the user 115 selects 142-9 the edit button 127. When the user 115 selects 142-3 the edit button 127, the user 115 is presented with an interface for modifying the selected image data 135, display data 149, and/or selfie image 143. For example, as shown in FIG. 1F-3, three different modifications to the image data 135 are presented. In first modified image data 191, the user 115 adds an overlay of to their image data 135. The overlay can include any personalized information. one or more options for sharing the captured image 135. In second modified image data 192, the user 115 merges or overlays the display data 149 (e.g., their fitness application display capture) with or over the image data 135. In third modified image data 193, the user 115 merges or overlays the display data 149 and the selfie image 143 with or over the image data 135. In some embodiments, the user 115 can edit the image data 135, display data 149, and/or selfie image 143 via one or more drawing tools. For example, as shown in FIG. 1F-4, the user 115 is able to draw free hand on the captured image data 135. In some embodiments, free hand text provided by the user 115 can be converted into typed text with user selected text. For example, as shown in FIG. 1F-5, the user's handwritten “Yes!” is converted into a typed text overlay. The above examples are non-exhaustive. A user 115 can edit the image data 135 in a number of different ways, such as adding a location, tagging one or more object, highlighting one or more portions of an image, merging different images, generating a slideshow, etc.

In FIG. 1G, the user 115 selects 142-3 the send button 121. When the user 115 selects 142-3 the send button 121, the user 115 is presented with one or more options for sharing the captured image 135. In some embodiment, the user 115 is able to select one or more of a messaging application, social media application, data transfer applications, etc. to share the captured image data. In some embodiments, selection of the send button 121 causes the wrist-wearable device 120 (or other device with a display 130) to present a contacts user interface 144 as shown in FIG. 1H.

The contacts user interface 144 can include one or more contacts (e.g., selectable contact user interface element 129) that the user 115 can select to send the captured image data 135. In some embodiments, the user 115 can select more than one contact to send the image data 135 to. In some embodiments, the image data 135 can be sent as a group message to a plurality of selected contacts. Alternatively, in some embodiments, the image data individually is sent to each selected contact. In some embodiments, the one or more contacts in the contacts user interface 144 are obtained via the one or more messaging applications, social media applications associated with the wrist-wearable device 120 or other device communicatively coupled with the wrist-wearable device 120. Alternatively or in addition, in some embodiments, the one or more contacts in the contacts user interface 144 are contacts that have been previously stored in memory (e.g., memory 860; FIGS. 8A-8B) of the wrist-wearable device 120.

FIG. 1I illustrates a user interface presented to the user 115 in response to selection of a contact in the contacts user interface 144. For example, FIG. 1I illustrates a user interface for Contact D 146 in response to user 115 selection 142-4 of the selectable contact user interface element 129 (which is associated with Contact D). In some embodiments, the user interface for a particular contact includes one or more applications that the user 115 and the contact have in common and/or have connected over. For example, the user interface for Contact D 146 includes an image sharing application 126-1, a media streaming or sharing application 126-2, a fitness application 126-3, and a messaging application 126-4. The user 115 can select at least one application that is used to share the image data 135 with. For example, as further shown in FIG. 1I, the user 115 provides an input (selection 142-5) identifying the messaging application as the application to be used in sharing the image data 135.

In FIG. 1J, displays a messaging thread user interface 147 associated with Contact D. In response to user selection 142-5 identifying the messaging application 1264-4 as the application to be used in sharing the image data 135, the wrist-wearable device 120 shares or transmits the image data to another user using the messaging application 1264-4. In some embodiments, message thread user interface 147 includes a history of the user 115's interaction with another user. For example, the message thread user interface 147 can include messages received from the other user (e.g., message user interface element 193 represented by the message “How's the run?”). The above example is non-limiting. Different applications include different user interfaces and allow for different actions be performed.

Although FIGS. 1E-1J illustrate the user 115 manually sharing the captured image data 135, in some embodiments, as described below in reference to FIGS. 1K-1N, the image data 135 can be automatically sent to another user. In particular, in some embodiments, the wrist-wearable device 120 can provide instructions to capture and send captured image data 135 to another user (specified by the user 115) when an image-capture trigger condition is satisfied. In some embodiments, the image data 135 can be automatically sent to another user to notify the other user that user 115 is en route to a target location. In some embodiments, the image data 135 can be automatically sent to another user as an additional security or safety measure. For example, the user 115 can define an image-capture trigger condition based on an elevated heart rate (e.g., above 180 BPM) or a particular location (e.g., a neighborhood with high crime rates), such that when the user 115's heart rate and/or position (measured by the sensors of the wrist-wearable device 120 and/or the head-wearable device 110) satisfy the image-capture trigger condition, the wrist-wearable device 120 provides instruction to capture and send image data 135 to another user, distinct from the user 115.

FIGS. 1K-1L illustrate automatically sharing the captured image data, in accordance some embodiments. In some embodiments, the user can opt-in to automatically sharing updates with other users. In some embodiments, a user 115 can associate the image-trigger capture condition with one or more contacts to share image data 135 with when captured. In some embodiments, the user 115 can also designate one or more contacts as part of a support or cheer group that receive updates as the user 115 is performing a physical activity. For example, as shown in FIG. 1K, the user 115 has a target hear rate between 120-150 BPM and a current hear rate of 100 BPM, and the wrist-wearable device 120 and/or head-wearable device 110 can contact one or more users in the user 115's support group to encourage the user 115. As shown in FIG. 1L, a message thread user interface 147 for contact D 146 shows the message 194 “Bob can use your support” along with a representation of image data 135 showing the user 115's current heart rate and target hear rate. This allows the user 115 and their selected support contacts to participate and encourage each other during different activities (e.g., a marathon, a century, a triathlon, an iron man challenge). In some embodiments, the one or more users in the user 115's support or cheer group are contacted when it is determined that the user 115 is no longer on pace to meet their target (e.g., the user started walking substantially reducing their hear rate, the user is running too fast running a risk of burning out, the user has stopped moving). For example, as shown in FIG. 1K, an image-trigger capture condition can be satisfied at point 180a (where the user stops moving) that causes the head-wearable device 110 to capture image data and send it to contact D as described above. This allows the user 115 to remain connected with their contacts and receive support when needed.

FIGS. 1M-1N illustrate one or more messages received and presented to the user during a physical activity, in accordance with some embodiments. In some embodiments, the user 115 can receive one or more messages that are presented via a display 130 of the wrist-wearable device and/or the head-wearable device 110. For example, as shown in FIGS. 1M and 1N, a message (You can do it Bob! Keep it up!) from the user's 115 friend, contact D, is presented via the wrist-wearable device 120 and the head-wearable device 110. In order to prevent interruptions during the performance of a physical activity, the user 115 can configure the wrist-wearable device 120 and/or the head-wearable device 110 to mute all incoming messages. In some embodiments, the user 115 is able to designate one or more user's that would not be muted. For example, a user 115 can select one or more users in their support or cheer group to always be unmuted.

FIGS. 1O-1P illustrate one or more responses that the user can provide to received messages during a physical activity, in accordance with some embodiments. In some embodiments, the user 115 can respond to one or more messages via the wrist-wearable device 120 and/or the head-wearable device 110. In some embodiments, the user can provide one or more handwritten symbols or gestures that are converted to quick and convenient messages. For example, as shown in FIG. 10, the user 115 draws a check mark on the display 130 of the wrist-wearable device that is converted as a thumbs up and shared with contact D (as shown in FIG. 1P). In some embodiments, EMG data and/or IMU data collected by the one or more sensors of the wrist-wearable device 120 can be used to determine one or more symbols, gestures, or text that a user 115 would like to respond with. For example, instead of drawing a check on the display 130 as shown in FIG. 10, the user 115 can perform a thumbs up gesture on the hand wearing the wrist-wearable device 120 and based on the EMG data and/or IMU data, a thumbs up gesture is sent to the receiving contact. Alternatively or in addition, in some embodiments, the user 115 can respond using the head-wearable device 110 and/or the wrist-wearable device 120 via voice to text, audio messages, etc.

Although FIGS. 1A-1P illustrate the coordination between the wrist-wearable device 120 and the head-wearable device 110 to determine, based on sensor data, whether an image-capture trigger condition is satisfied and the capture of image data, intermediary devices communicatively coupled with the head-wearable device 110 and/or the wrist-wearable device 120 (e.g., smartphones 874a, tablets, laptops, etc.) can be used to determine whether an image-capture trigger condition is satisfied and/or capture image data 135.

FIG. 2 illustrates a flow diagram of a method for using sensor data from a wrist-wearable 120 device to monitor image-capture trigger conditions for determining when to capture images using an imaging device of a head-wearable device 110, in accordance with some embodiments. The head-wearable device and wrist-wearable device are worn by a user. Operations (e.g., steps) of the method 200 can be performed by one or more processors (e.g., central processing unit and/or MCU; processors 850, FIGS. 8A-8B) of a head-wearable device 110. In some embodiments, the head-wearable device 110 is coupled with one or more sensors (e.g., various sensors discussed in reference to FIGS. 8A-8B, such as a heart rate sensor, IMU, an EMG sensor, SpO2 sensor, altimeter, thermal sensor or thermal couple, ambient light sensor, ambient noise sensor), a display, a speaker, an image device (FIGS. 8A-8B; e.g., a camera), and a microphone to perform the one or more operations. At least some of the operations shown in FIG. 2 correspond to instructions stored in a computer memory or computer-readable storage medium (e.g., storage, ram, and/or memory 860, FIGS. 8A-8B). Operations of the method 200 can be performed by the head-wearable device 110 alone or in conjunction with one or more processors and/or hardware components of another device communicatively coupled to the head-wearable device 110 (e.g., a wrist-wearable device 120, a smartphone 874a, a laptop, a tablet, etc.) and/or instructions stored in memory or computer-readable medium of the other device communicatively coupled to the head-wearable device 110.

The method 200 includes receiving (210) sensor data from an electronic device (e.g., wrist-wearable device 120) communicatively coupled to a head-wearable device 110. The method 200 further includes determining (220) whether the sensor data indicates that an image-capture trigger condition for is satisfied. For example, as described above in references to FIG. 1A-1B-3, the head-wearable device 110 can receive sensor data indicating that the user 115 is performing a running activity as well as their position, which is used to determine whether an image-capture trigger condition (e.g., user 115's position at a target destination 181; FIGS. 1A-1B-3) is satisfied.

In accordance with the determination that the received sensor data does not satisfy an image-capture trigger condition (“No” at operation 220), the method 200 returns to operation 210 and waits to receive additional sensor data from an electronic device communicatively coupled with the head-wearable device 110. Alternatively, in accordance with a determination that the received sensor data does satisfy an image-capture trigger condition (“Yes” at operation 220), the method further includes instructing (230) an imaging device communicatively coupled with the head-wearable device 110 to capture image data 135. For example, as further described above in reference to FIG. 1B-1-1B-3, when the user 115 has reaches the target destination satisfying an image-capture trigger condition, the imaging device 128 of the head-wearable device 110 is caused to capture image data 135. In some embodiments, after the image data is captured, the method 200 includes instructing (235) a display communicatively coupled with the head-wearable device presents a representation of the image data 135. For example, as shown above in reference to FIG. 1E, a representation of the image data 135 captured by the imaging device 128 of the head-wearable device 110 is caused to be presented at a display 130 of the wrist-wearable device 120.

In some embodiments, the method 200 further includes determining (240) whether the captured image data should be shared with one or more users. In some embodiments, a determination that the captured image data should be shared with one or more users is based on user input. In particular, a user can provide one or more inputs at the head-wearable device 110, wrist-wearable device 120, and/or an intermediary device communicatively coupled with the head-wearable device 110, that cause the head-wearable device 110 and/or another communicatively coupled electronic device (e.g., the wrist-wearable device 120) to share the image data with at least one other device. As shown in FIGS. 1G-1N, the user 115 can provide one or more inputs at the wrist-wearable device 120 identifying image data 135 to be sent, a recipient of the image data 135, an application to be used in sharing the image data, and/or other preferences.

In some embodiments, in accordance with a determination that the image data should be shared with one or more users (“Yes” at operation 240), the method 200 further includes instructing (250) the head-wearable device 120 (or an electronic device communicatively coupled with the head-wearable device 110) to send the image data to respective electronic devices associated with the one or more users. For example, in FIG. 1I, the user 115 selects the option to send the captured image 135 to a contact via a messaging application, and, in FIG. 1J, the image data 135 is sent to the selected contact using the messaging application. After sending the image to the respective electronic devices associated with the one or more users, the method 200 returns to operation 210 and waits to receive additional sensor data from an electronic device communicatively coupled with the head-wearable device 110.

Returning to operation 240, in accordance with a determination that the image data should not be shared with one or more users (“No” at operation 240), the method 200 returns to operation 210 and waits to receive additional sensor data from an electronic device communicatively coupled with the head-wearable device 110.

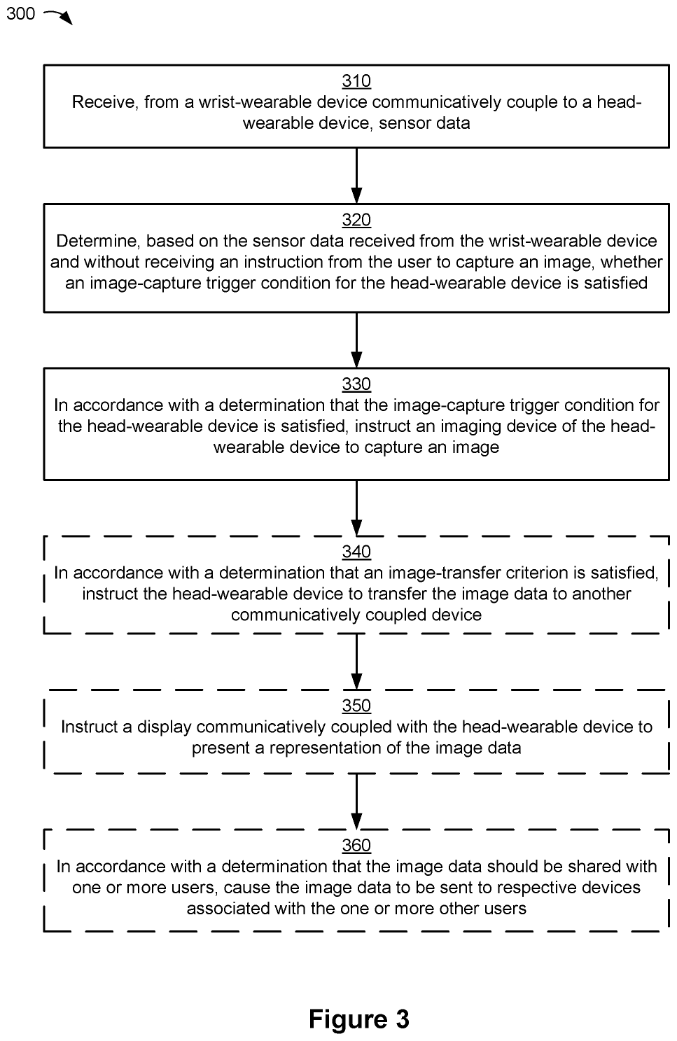

FIG. 3 illustrates a detailed flow diagram of a method of using sensor data from a wrist-wearable device to monitor image-capture trigger conditions trigger conditions for determining when to capture images using an imaging device of a head-wearable device, in accordance with some embodiments. The head-wearable device and wrist-wearable device are worn by a user. Similar to method 200 of FIG. 2, operations of the method 300 can be performed by one or more processors of a head-wearable device 110. At least some of the operations shown in FIG. 3 correspond to instructions stored in a computer memory or computer-readable storage medium. Operations of the method 300 can be performed by the head-wearable 110 alone or in conjunction with one or more processors and/or hardware components of another device (e.g., a wrist wearable device 120 and/or an intermediary device described below in reference to FIGS. 8A-8B) communicatively coupled to the head-wearable device 110 and/or instructions stored in memory or computer-readable medium of the other device communicatively coupled to the head-wearable device 110.

Method 300 includes receiving (310), from a wrist-wearable device 120 communicatively coupled to a head-wearable device 110, sensor data. In some embodiments, the sensor data received from the wrist-wearable device 120 is from a first type of sensor and the head-wearable device 110 does not include the first type of sensor. Therefore, the head-wearable device 110 is able to benefit from sensor-data monitoring capabilities that it does not possess. As a result, certain head-wearable devices 110 can remain lighter weight and thus have a more acceptable form factor that consumers will be more willing to accept and wear in normal use cases; can also include fewer components fewer components that could potentially fail; and can make more efficient use of limited power resources. As one example, the wrist-wearable device 120 can include a global-positioning sensor (GPS), which the head-wearable device 110 might not possess. Other examples include various types of biometric sensors that might remain only at the wrist-wearable device 120 (or other electronic device used for the hardware-control operations discussed herein), which biometric sensors can include one or more of heartrate sensors, SpO2 sensors, blood-pressure sensors, neuromuscular-signal sensors, etc.

The method 300 includes, determining (320), based on the sensor data received from the wrist-wearable device 120 and without receiving an instruction from the user to capture an image, whether an image-capture trigger condition for the head-wearable device 110 is satisfied. Additionally or alternatively, in some embodiments, a determination that the image-capture trigger condition is satisfied is based on sensor data from one or more sensors of the head-wearable device 110. In some embodiments, a determination that an image-capture trigger condition is based on identifying, using data from one or both of the imaging device of the head-wearable device or an imaging device of the wrist-wearable device, a predefined object (e.g., a type of image-capture trigger condition as described below) within a field of view of the user. For example, computer vison can be used to assist in determining whether an image-capture trigger condition is satisfied. In some embodiments, one or more transient images (e.g., images temporarily saved in memory and discarded after analysis (e.g., no longer than minute)) captured by the imaging device of the head-wearable device 110 (or imaging device of the electronic device) can be analyzed to assist in determining whether an image-capture trigger condition is satisfied.

In some embodiments, an image-capture trigger condition can include a predefined heart rate, a predefined location, a predefined velocity, a predefined duration at which an event occurs (e.g., performing a physical activity for fifteen minutes), a predefined distance. In some embodiments, an image-capture trigger condition includes predefined objects such as a particular mile marker on the side of the road, a landmark object (e.g., a rock formation), signs placed by an organizer of an exercise event (signs at a water stop of a footrace), etc. In some embodiments, an image-capture trigger condition is determined based on the user activity and/or user data. For example, an image-capture trigger condition can be based on a user 115's daily jogging route, average running pace, personal records, frequency at which different objects are within a field of view of an imaging device of the head-wearable device 110, etc. In some embodiments, an image-capture trigger condition is user defined. In some embodiments, more than one image-capture trigger condition can be used.

As non-exhaustive examples, an image-capture trigger condition can be determined to be satisfied based on a user 115's hear rate, sensed by one or more sensors of the wrist-wearable device 120, reaching a target heartrate; the user 115 traveling a target distance during an exercise activity which is monitored in part with the sensor data of the wrist-wearable device 120; the user 115 reaching a target velocity during an exercise activity which is monitored in part with the sensor data of the wrist-wearable device 120; the user 115's monitored physical activity lasting a predetermined duration; image recognition (e.g., analysis performed on an image captured by the wrist-wearable device 120 and/or the head-wearable device 110) performed on image data; a position of the wrist-wearable device 120 and/or a position of the head-wearable device 110 detected in part using the sensor data (e.g., staring upwards to imply the user 115 is looking at something interesting); etc. Additional examples of the image-capture trigger conditions are provided above in reference to FIGS. 1A-1D.

The method 300 further includes, in accordance with a determination that the image-capture trigger condition for the head-wearable device 110 is satisfied, instructing (330) an imaging device of the head-wearable device 110 to capture an image. The instructing operation can occur very shortly after the determination is made (e.g., within 2 ms of the determination), and the instructing operation can also occur without any further user 115 instruction to capture the image (e.g., the system proceeds to capture the image because the image-capture trigger was satisfied and does not need to receive any specific user request beforehand). In some embodiments, instructing the imaging device 128 of the head-wearable device 110 to capture the image data includes instructing the imaging to capture a plurality of images. Each of the plurality of images can be stored in a common data structure or at least be associated with one another for easy access and viewing later on. For example, all of the captured images can be stored in the same album or associated with the same event. In an additional example, at least two images can be captured when the user 115 reaches a particular landmark. Each image is associated with the same album such that the user 115 can select their favorite. Alternatively, all images captured during a particular event can be associated with one another (e.g., 20 images captured during one long run long will be placed in the same album). Examples of the captured image data are provided above in reference to FIG. 1D.

In some embodiments, additional sensor data is received from the wrist-wearable device 120 that is communicatively coupled to the head-wearable device 110, and the method 300 includes determining, based on the additional sensor data received from the wrist-wearable device 120, whether an additional image-capture trigger condition for the head-wearable device 110 is satisfied. The additional image-capture trigger condition can be distinct from the image-capture trigger condition, and in accordance with a determination that the additional image-capture trigger condition for the head-wearable device 110 is satisfied, the method 300 further includes instructing the imaging device of the head-wearable device 110 to capture an additional image. Thus, multiple different image-capture trigger conditions can be monitored and used to cause the head-wearable device 110 to capture images at different points in time dependent on an evaluation of the pertinent sensor data from the wrist-wearable device 120.

In some embodiments, in accordance with the determination that the image-capture trigger condition is satisfied, the method 300 includes instructing the wrist-wearable device 120 to store information concerning the user's performance of an activity for association with the image captured using the imaging device of the head-wearable device 110. For example, if the user 115 is using a fitness application that is tracking the user's workout, the trigger can cause the electronic device to store information associated with the physical activity (e.g., hear rate, oxygen saturation, body temperature, burned calories) and/or capture a screenshot of the information displayed via the fitness application. In this way, the user 115 has a record of goals that can be shared with their friends, images that can be combined or linked together, images that can be overlaid together, etc. In some embodiments, the wrist-wearable device is instructed to capture a screenshot of a presented display substantially simultaneously (e.g., within 0 s-15 ms, no more than 1 sec, etc.) with the image data captured by the imaging device of the head-worn wearable. Examples of the captured display data are provided above in reference to FIG. 1B-2.

In some embodiment, in accordance with the determination that the image-capture trigger condition is satisfied, the method 300 includes instructing the wrist-wearable device 120 and/or the head-wearable device 110 to present a notification to the user 115 requesting for personal image or “selfie.” The user 115 can respond to the notification (e.g., via a user input), which activates an imaging device 128 on the wrist-wearable device 120. The imaging device 128 of the wrist-wearable device 120 can capture an image of the user 115 once the user 115's face is in the field of view of the imaging device of the wrist-wearable device 120 and/or the user manually initiates capture of the image data. Alternatively, in some embodiments, the imaging device of the wrist-wearable device is instructed to capture an image substantially simultaneously with the image data captured by the imaging device of the head-wearable device. In some embodiments, the notification can instruct the user to position the wrist-wearable device 120 such that it is oriented towards a face of the user.

In some embodiments, in accordance with the determination that the image-capture trigger condition for the head-wearable device 110 is satisfied, instructing an imaging device of the wrist-wearable device 120 to capture another image, and in accordance with the determination that the additional image-capture trigger condition for the head-wearable device 110 is satisfied, forgoing instructing the imaging device of the wrist-wearable device 120 to capture an image. For example, some of the image-capture trigger conditions can cause multiple devices to capture images, such as images captured by both the head-wearable device 110 and the wrist-wearable device 120, whereas other image-capture trigger conditions can cause only one device to capture an image (e.g., one or both of the head-wearable device 110 and wrist-wearable device 120).

The different images captured by the wrist-wearable device 120 and/or the head-wearable device 110 allow the user to further personalize the image data automatically captured in response to satisfaction of image-capture trigger condition. For example, the user 115 can collate different images captured while the user participated in a running marathon, which would allow the user 115 to create long lasting memories of the event that can be shared with others. In some embodiments, certain of the image-capture trigger conditions can be configured such that the device that is capturing the image should be oriented a particular way and the system can notify (audibly or visually or via haptic feedback, or combinations thereof) the user to place the device in the needed orientation (e.g., orient the wrist-wearable device to allow for capturing a selfie of the user while exercising, which can be combined with an image of the user's field of view that can be captured via the imaging device of the head-wearable device).

In some embodiments, the method 300 includes, in accordance with a determination that an image-transfer criterion is satisfied, instructing (340) the head-wearable device to transfer the image data to another communicatively coupled device (e.g., the wrist-wearable device 120). For example, the head-wearable device 110 can transfer the captured image data to the wrist-wearable device 120 to display a preview of the captured image data. For example, a user 115 could take a photo using the head-wearable device 110 and send it to a wrist-wearable device 120 before sharing it with another user 115. In some embodiments, a preview on the wrist-wearable device 120 is only presented after the wrist of the user 115 is tilted (e.g., with the display 130 towards the user 115. In some embodiments, the head-wearable device 110 can store the image before sending it to the wrist-wearable device 120 for viewing. In some embodiments, the head-wearable device 110 deletes stored image data after successful transfer of the image data to increase the amount of available memory.

The image-transfer criterion can include the occurrence of certain events, predetermined locations, predetermined biometric data, a predetermined velocity, image recognition, etc. For example, the head-wearable device 110 can determine that an image-transfer criterion is satisfied due in part to the user 115 of the wrist-wearable device 120 completing or pausing an exercise activity. In another example, the head-wearable device 110 can transfer the image data once the user 115 stops, slows down, reaches a rest point, or pauses the workout. This reduces the number of notifications that the user 115 receives, conserves battery life by reducing the number of transfers that need to be performed before a successful transfer occurs, etc. Additional examples of image-transfer criteria are provided above in reference to FIGS. 1C and 1D.

In some embodiments, the method 300 further includes instructing (350) a display communicatively coupled with the head-wearable device to present a representation of the image data. For example, as shown above in reference to FIG. 1D, image data captured by the head-wearable device 110 can be presented to the user 115 via a display 130 of the wrist-wearable device 120. In some embodiments, after the image is caused to be sent for display at the wrist-wearable device 120, the image data is stored at the wrist-wearable device 120 and removed from the head-wearable device 110. This feature makes efficient use of limited power and computing resources of the head-wearable device 110 since once the image is offloaded to another device, it can then be removed from the storage of the head-wearable device 110 and free up the limited power and computing resources of the head-wearable device 110 for other functions, while also furthering the goal of ensuring that the head-wearable device 110 can maintain a light-weight socially acceptable form factor.

In some embodiments, after the image is captured, the method 300 further determines, in accordance with a determination that the image data should be shared with one or more users, causing (360) the image data to be sent to respective devices associated with the one or more other users. In some embodiments, before causing the image data to be sent to the respective devices associated with the one or more other users, the method 300 includes applying one or more of an overlay (e.g., can apply a heart rate to the captured image, a running or completion time, a duration, etc.), a time stamp (e.g., when the image was captured), geolocation data (e.g., where the image was captured), and a tag (e.g., a recognized location or person that the user 115 is with) to the image to produce a modified image that is then caused to be sent to the respective devices associated with the one or more other users. For example, the user 115 might want to share their running completion time with another user 115 to share that the user 115 has achieved a personal record.

In some embodiments, before causing the image to be sent to the respective devices associated with the one or more other users, the method 300 includes causing the image to be sent for display at the wrist-wearable device 120 within an image-selection user interface, wherein the determination that the image should be shared with the one or more other users is based on a selection of the image from within the image-selection user interface displayed at the wrist-wearable device 120. For example, the user 115 could send the image to the wrist-wearable device 120 so the user 115 could more easily select the image and send it to another user. Different examples of the user interfaces for sharing the captured image data are provided above in reference to FIGS. 1G-1N.

In some embodiments, the user 115 can define or more image-sharing condition, such that when the image-sharing condition is satisfied, captured image data is sent to one or more users. For example, in some embodiments, the determination that the image should be shared with one or more other users is made when it is determined that the user 115 has decreased their performance during an exercise activity. Thus, the images can be automatically shared with close friends to help motivate the user 115 to reach exercise goals, such that when their performance decreases (e.g., pace slows below a target threshold pace such as 9 minutes per mile for a run or 5 minutes per mile for a cycling ride), then images can be shared to the other users so that they can provide encouragement to the user 115. The user 115 selection to send the captured image can be received from the head-wearable device 110 or another electronic device communicatively coupled to the head-wearable device 110. For example, the user 115 could nod to choose an image to share or provide an audible confirmation.

While the primary example discussed herein relates to use of sensor data from a wrist-wearable device to determine when to capture images using an imaging device of a head-wearable device, other more general example use cases are also contemplated. For instance, certain embodiments can make use of sensor data from other types of electronic devices, such as smartphones, rather than, or in addition to, the sensor data from a wrist-wearable device. Moreover, the more general aspect of controlling hardware at the head-wearable device based on sensor data from some other electronic device is also recognized, such that other hardware features of the head-wearable device can be controlled based on monitoring of appropriate trigger conditions. These other hardware features can include, but are not limited to, control of a speaker of the head-wearable device, e.g., by starting or stopping music (and/or specific songs or podcasts, and/or controlling audio-playback functions such as volume, bass level, etc.) based on a predetermined rate of speed measured based on sensor data from the other electronic device while the user is exercising; controlling illumination of a light source of the head-wearable device (e.g., a head-lamp or other type of coupled light source for the head-wearable device based on the exterior lighting conditions detected based on sensor data from the other electronic device, activating a display 130 to provide directions or a map to the user, etc.

In certain embodiments or circumstances, head-wearable devices can include a camera and a speaker, but may not include a full sensor package like that found in wrist-wearable devices or other types of electronic devices (e.g., smartphones). Thus, it can be advantageous to utilize sensor data from a device that has the sensors (e.g., the wrist-wearable device) to create new hardware-control triggers for the head-wearable device (e.g., to control a camera of the head-wearable device as the user reaches various milestones during an exercise routine, as the user's reaches favorite segments or locations during a run (e.g., a picture can be captured at a particular point during a difficult hill climb), and/or to motivate the user (e.g., captured pictures can be shared immediately with close friends who can then motivate the user to push themselves to meet their goals; and/or music selection and playback characteristics can be altered to motivate a user toward new exercise goals).

In some embodiments, enabling the features to allow for controlling hardware of the head-wearable device based on sensor data from another electronic device is done after a user opt-in process, which includes the user providing affirmative consent to the collection of sensor data to assist with offering these hardware-control features (e.g., which can be provided while setting up one or both of the head-wearable device and the other electronic device, and which can be done via a settings user interface). Even after opt-in, users are, in some embodiments, able to opt-out at any time (e.g., by accessing a settings screen and disabling the pertinent features).

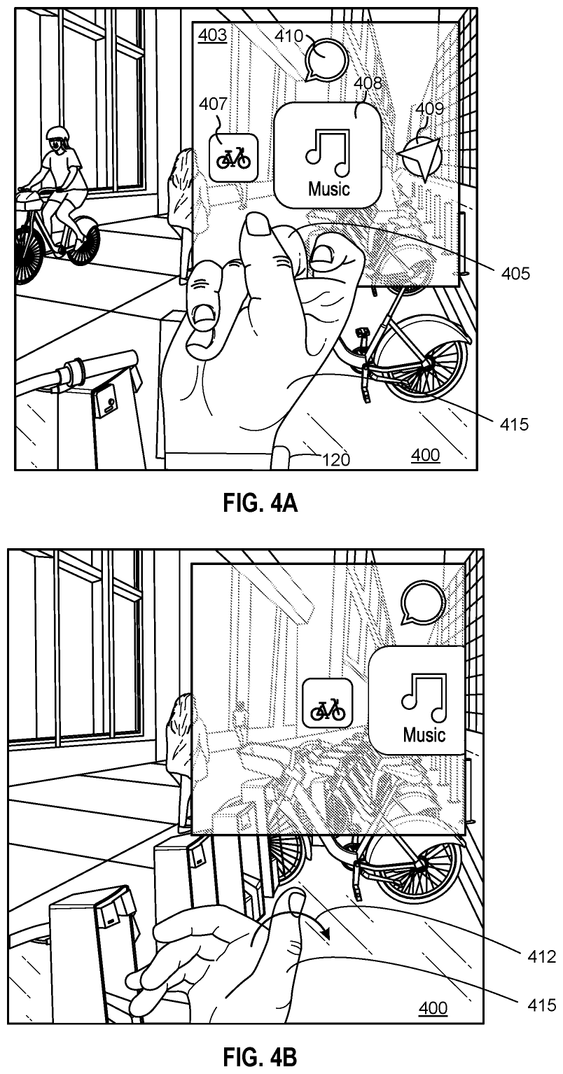

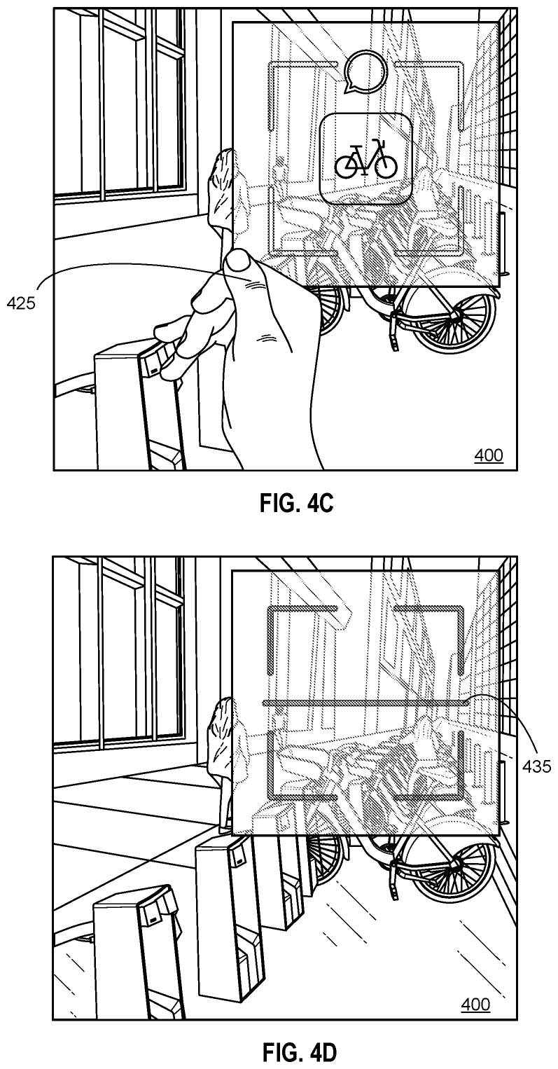

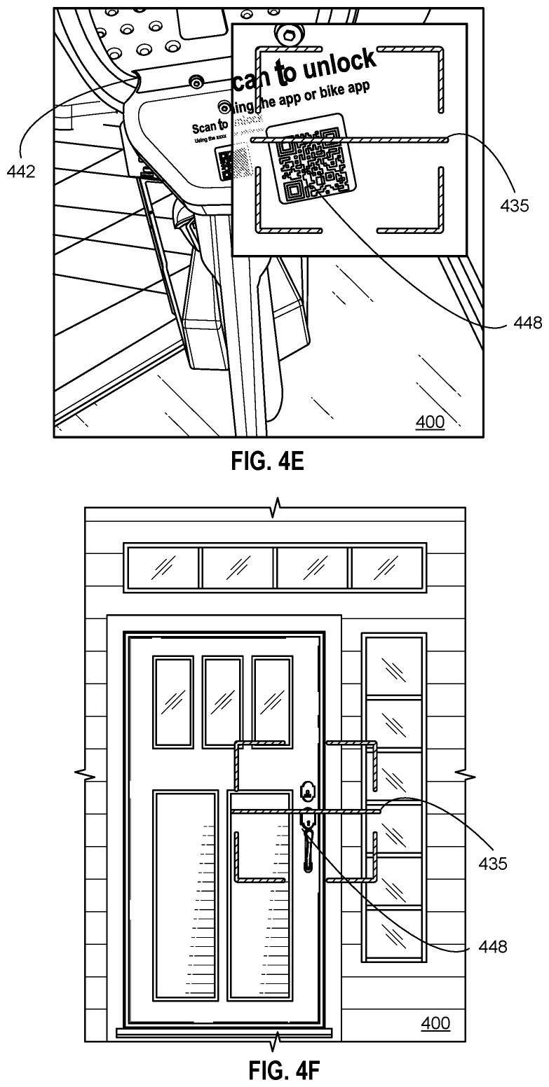

FIGS. 4A-4F illustrate using sensor data from a wrist-wearable device to activate a communicatively coupled head-wearable device, in accordance with some embodiments. In particular, using sensor data from the wrist-wearable device 120 worn by a user 415 (e.g., represented by user's hand) to activate and/or initiate one or more applications or operations on the head-wearable device 110 (e.g., FIG. 1A) also worn by the user 415. For example, the wrist-wearable device 120, while worn by the user 415, can monitor sensor data captured by one or more sensor (e.g., EMG sensors) of the wrist-wearable device 120, and the sensor data can be used to determine whether the user 415 performed an in-air hand gesture associated with one or more applications or operations on the head-wearable device 110. Additionally or alternatively, in some embodiments, the head-wearable device 110, worn by the user 415, can monitor image data, via a communicatively coupled imaging device 128 (e.g., FIG. 1A), and determine whether the user 415 performed an in-air hand gesture associated with one or more applications or operations on the head-wearable device 110. In some embodiments, the determination that the user 415 performed an in-air hand gesture is determined by wrist-wearable device 120, the head-wearable device 110, and/or a communicatively coupled intermediary device. For example, the sensor data captured by one or more sensor of the wrist-wearable device 120 can be provided to an intermediary device (e.g., a portable computing unit) that determines, based on the sensor data, that the user 415 performed an in-air hand gesture.

Turning to FIG. 4A, the user 415's field of view 400 while wearing the head-wearable device 110 is shown. The head-wearable device 110 is communicatively coupled to the wrist-wearable device 120 such that the head-wearable device 110 can cause the performance of one or more operations at the wrist-wearable device 120, and/or vice versa. For example, sensor data received from the wrist-wearable device 120 worn by the user 415 indicating performance of an in-air hand gesture associated an operation (e.g., unlocking access to a physical item, such as a rentable bicycle) can cause the head-wearable device 110 to perform the operation or a portion of the operation (e.g., initiating an application for unlocking access to the physical item).

In some embodiments, a hand gesture (e.g., in-air finger-snap gesture 405) performed by the user 415 and sensed by the wrist-wearable device 120 causes the head-wearable device 110 to present an AR user interface 403. The AR user interface 403 can include one or more user interface elements associated with one or more applications and/or operations that can be performed by the wrist-wearable device 120 and/or head-wearable device 110. For example, the AR user interface 403 includes a bike-rental application user interface element 407, a music application user interface element 408, a navigation application user interface element 409, and a messaging application user interface element 410. The AR user interface 403 and the user interface elements can be presented within the user 415's field of view 400. In some embodiments, the AR user interface 403 and the user interface elements are presented in a portion of the user 415's field of view 400 (e.g., via a display of the head-wearable device 110 that occupies a portion, less than all, of a lens or lenses). Alternatively, or in addition, in some embodiments, the AR user interface 403 and the user interface elements are presented transparent or semi-transparent such that the user 415's vision is not hindered.

The user 415 can perform additional hand gestures that, when sensed by the wrist-wearable device 120, cause a command to be performed at the head-wearable device 110 and/or the wrist-wearable device 120. For example, as shown in FIG. 4B, the user 115 performs an in-air thumb-roll gesture 412 to browse different applications presented by the head-wearable device 110 (e.g., as shown by the AR user interface 403 switching or scrolling from the music application user interface element 408 to the bike-rental application user interface element 407). Further, as shown in FIG. 4C, the user 115 performs yet another hand gesture (in-air thumb-press gesture 425) to select an application (e.g., user input selecting the bike-rental application user interface element 407).

Turning to FIG. 4D, the bike-rental application is initiated in response to the user 415's selection. The bike-rental application is presented within the AR user interface 403 and can be used to unlock access to a physical item (e.g., a bicycle). In some embodiments, an application to unlock access to a physical item includes using image data captured via an imaging device 128 to determine that an area of interest in the image data satisfies an image-data-searching criteria. The image-data-searching criteria can include detection of a visual identifier (e.g., a QR code, a barcode, an encoded message, etc.); typed or handwritten characters (in any language); predetermined object properties and/or characteristics (e.g., product shapes (e.g., car, bottle, etc.), trademarks or other recognizable insignia, etc.). In some embodiments, a visual identifier assists the user in accessing additional information associated with the visual identifier (e.g., opening a URL, providing security information, etc.). In some embodiments, the typed or handwritten characters can include information that can be translated for the user; terms, acronyms, and/or words that can be defined for the user; and/or characters or combination of terms that can be searched (e.g., via a private or public search engine).

As shown between FIGS. 4C and 4D, in response to a determination that the in-air thumb-press gesture 425 was performed, an imaging device 128 of a head-wearable device is activated and captures image data, which is used to determine whether an area of interest in the image data satisfies an image-data-searching criteria. While the imaging device 128 captures image data, a representation of the image data can be presented to the user 415 via the AR user interface 403. The area of interest can be presented to the user 415 as a crosshair user interface element 435 to provide the user with a visual aid for pointing or aiming the imaging device 128. For example, the crosshair user interface element 435 can be presented as bounding box including a center line for aligning a visual identifier. In some embodiments, the crosshair user interface element 435 is presented in response to a user input to initiate an application to unlock access to a physical item via the wrist-wearable device 120 and/or the head-wearable device 110. Alternatively, the user 415 can toggle presentation of the crosshair user interface element 435. In some embodiments, the user 415 can adjust the appearance of the crosshair user interface element 435 (e.g., change the shape from a square to a triangle, changing a size of the crosshair, changing a color of the crosshair, etc.). In this way, the user 415 can customize the crosshair user interface element 435 such that it is not distracting and/or personalized.

A determination that an area of interest in the image data satisfies an image-data-searching criteria can be made while the image data is being captured by an imaging device 128. For example, as shown in FIG. 4E, while the bike-rental application is active and the imaging device 128 captures image data, the user 415 approaches a bicycle docking station 442, which includes a visual identifier 448 (e.g., a QR code) for unlocking access to a bicycle, and attempts to align the crosshair user interface element 435 with the visual identifier 448. While the user 415 attempts to align the crosshair user interface element 435 with the visual identifier 448, the crosshair user interface element 435 can be modified to notify the user 415 that the visual identifier 448 is within an area of interest in the image data and/or the visual identifier 448 within the area of interest in the image data satisfies an image-data-searching criteria. For example, the crosshair user interface element 435 can be presented in a first color (e.g., red) and/or first shape (e.g., square) when the visual identifier 448 is not within an area of interest in the image data and presented in a second color (e.g., green) and/or second shape (e.g., circle) when the visual identifier 448 is within the area of interest in the image data.

In some embodiments, while the image data is being captured by an imaging device 128, the imaging device 128 can be adjusted and/or the image data can be processed to assist the user 415 in aligning the crosshair user interface element 435 or satisfying the image-data-searching criteria of the area of interest in the image data. For example, as further shown in FIG. 4E, the image data is processed to identify the visual identifier 448 and the imaging device 128 focuses and/or zooms-in at the location of the visual identifier 448. In some embodiments, a determination that the area of interest satisfies the image-data-searching criteria is made after a determination that the captured image data is stable (e.g., imaging device is not shaking moving, rotating, etc.), the head-wearable device 110 and/or wrist-wearable device 120 have a predetermined position (e.g., the head-wearable device 110 has a downward position such that the imaging device is pointing to down to a specific object), and/or the user 415 provided an additional input to detect one or more objects within a portion of the captured image data.

In accordance with a determination that the area of interest satisfies the image-data-searching criteria, the wrist-wearable device 120 and/or the head-wearable device 110 identifies and/or processes a portion of the image data. For example, in accordance with a determination that the visual identifier 448 is within the area of interest, information associated with the visual identifier 488 is retrieved and/or accessed for the user 415. In some embodiments, the visual identifier 488 can be associated with a user account or other user identifying information. For example, in FIG. 4E, after the visual identifier 448 is detected within the area of interest, information corresponding to the visual identifier 448 is accessed, and user information is shared. In particular, a bicycle associated with the bike-rental application is identified and user information for unlocking access to the bicycle (e.g., login credentials, payment information, etc.) is shared with the bike-rental application. In this way, the user can quickly gain access to a physical object without having to manually input their information (e.g., the user 415 can gain access to the physical object with minimal inputs through the use of wearable devices). In some embodiments, the user 415 can be asked to register an account or provide payment information if the application for unlocking access to a physical object has not been used before or if the user's login information is not recognized or accepted.

Alternatively, in accordance with a determination that the area of interest does not satisfy the image-data-searching criteria, the wrist-wearable device 120 and/or the head-wearable device 110 can prompt the user 415 to adjust a position of the imaging device 128 and/or collect additional image data to be used in a subsequent determination. The additional image data can be used to determine whether the area of interest satisfies the image-data-searching criteria.

FIG. 4F shows an alternate example of unlocking access to a physical object. In particular, FIG. 4F shows the user 415 unlocking access to a door of their house. The door can include a visual identifier 448 that can be used to identify the door (or residence), the users associated with the door, and/or user's able to gain access to a residence via the door.

While the above example describe unlocking access to a physical object, the skilled artisan will appreciate upon reading the descriptions that user inputs can be used to initiate other applications of the wrist-wearable device 120 and/or the head-wearable device 110. For example, user inputs that the wrist-wearable device 120 can cause the head-wearable device 110 to open music application, a messaging application, and/or other applications (e.g., gaming applications, social media applications, camera applications, web-based applications, financial applications, etc.). Alternatively, user inputs that the head-wearable device 110 can cause the wrist-wearable device 120 to open music application, a messaging application, and/or other applications.

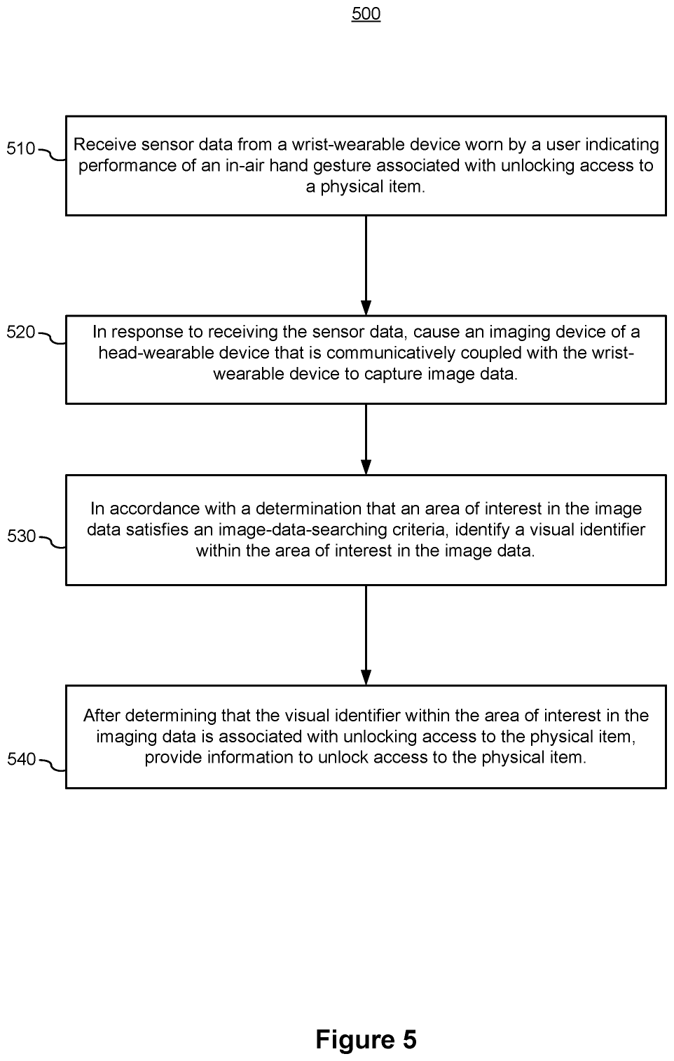

FIG. 5 illustrates a detailed flow diagram of a method of unlocking access to a physical item using a combination of a wrist-wearable device and a head-wearable device, in accordance with some embodiments. The head-wearable device and wrist-wearable device are example wearable devices worn by a user (e.g., head-wearable device 110 and wrist-wearable device 120 described above in reference to FIGS. 1A-4F). The operations of method 500 can be performed by one or more processors of a wrist-wearable device 120 and/or a head-wearable device 110. At least some of the operations shown in FIG. 5 correspond to instructions stored in a computer memory or computer-readable storage medium. Operations of the method 500 can be performed by the wrist-wearable device 120 alone or in conjunction with one or more processors and/or hardware components of another device (e.g., a head-wearable device 110 and/or an intermediary device described below in reference to FIGS. 8A-8B) communicatively coupled to the wrist-wearable device 120 and/or instructions stored in memory or computer-readable medium of the other device communicatively coupled to the wrist-wearable device 120.

The method 500 includes receiving (510) sensor data from a wrist-wearable device worn by a user indicating performance of an in-air hand gesture associated with unlocking access to a physical item. For example, as shown and described above in reference to FIG. 4A, a user can perform an in-air finger-snap gesture 405 to cause a wearable device to present an user interface for selecting one or more applications. Alternatively, the user can perform an in-air hand gesture that directly initiates an application for unlocking access to a physical item.

The method 500 includes, in response to receiving the sensor data, causing (520) an imaging device of a head-wearable device that is communicatively coupled with the wrist-wearable device to capture image data. For example, as shown and described above in reference to FIG. 4E, an imaging device of the head-wearable device is activated to capture image data for unlocking access to a physical item. The method 500 includes, in accordance with a determination that an area of interest in the image data satisfies an image-data-searching criteria, identifying (530) a visual identifier within the area of interest in the image data. For example, as further shown and described above in reference to FIG. 4E, a crosshair user interface element 435 (representative of the area of interest) is presented to the user, via a display of the head-wearable device, such that the user can align the crosshair user interface element 435 with a QR code. Further, the method 500 includes, after determining that the visual identifier within the area of interest in the imaging data is associated with unlocking access to the physical item, providing (540) information to unlock access to the physical item. For example, the QR code within the crosshair user interface element 435 can be processed and information with the QR code can be accessed (e.g., type of service, payment request, company associated with the QR code, user account look up, etc.) and/or user information associated with the QR code can be shared (e.g., user ID, user password, user payment information, etc.).

In some embodiments, the method 500 includes, before the determination that the area of interest in the image data satisfies the image-data-searching criteria is made, presenting the area of interest in the image data at the head-wearable device as zoomed-in image data. For example, as shown and described above in reference to FIG. 4E, a portion of the image data within the crosshair user interface element 435 is zoomed-in or magnified to assist the user in the capture of the visual identifier. In some embodiments, the visual identifier is identified within the zoomed-in image data. In some embodiments, the visual identifier includes one or more of a QR code, a barcode, writing, a label, and an object identified by an image-recognition algorithm, etc.

In some embodiments, the area of interest in the image data is presented with an alignment marker (e.g., crosshair user interface element 435), and the image-data-searching criteria is determined to be satisfied when it is determined that the visual identifier is positioned with respect to the alignment marker. In some embodiments, the determination that the area of interest in the image data satisfies the image-data-searching criteria is made is in response to a determination that the head-wearable device is positioned in a stable downward position.

In some embodiments, the method 500 includes, before identifying the visual identifier, and in accordance with a determination that an additional area of interest in the image data fails to satisfy the image-data-searching criteria, forgoing identifying a visual identifier within the additional area of interest in the image data. In other words, the processing logic can be configured to ignore certain areas of interest in the image data and to focus only on the areas of interest that might have content associated with unlocking access to the physical item. Alternatively or in addition, in some embodiments, the method 500 includes, before determining that the visual identifier within the area of interest in the image data is associated with unlocking access to the physical item, and in accordance with a determination that the visual identifier is not associated with unlocking access to the physical item, forgoing providing information to unlock access to the physical item.

In some embodiments, the method 500 includes, in response to receiving a second sensor data, causing the imaging device of the head-wearable device that is communicatively coupled with the wrist-wearable device to capture second image data. The method 500 further includes, in accordance with a determination that a second area of interest in the second image data satisfies a second image-data-searching criteria, identifying a second visual identifier within the second area of interest in the second image data; and after determining that the second visual identifier within the second area of interest in the second image data is associated with unlocking access to a second physical item, providing second information to unlock access to the second physical item. For example, as shown and described above in reference to FIG. 4F, the captured image data can be used to unlock the user's front door. Additional non-limiting examples of physical items that can be unlocked include rental cars, lock boxes, vending machines, scooters, books, etc.

Although the above examples describe access unlocking access to a physical item, the disclosed method can also be used to provide user info to complete a transaction (e.g., account information, verification information, payment information, etc.), image and/or information lookup (e.g., performing a search of an object within the image data (e.g., product search (e.g., cleaning product look up), product identification (e.g., type of car), price comparisons, etc.), word lookup and/or definition, language translation, etc.

### Example Wrist-Wearable Devices

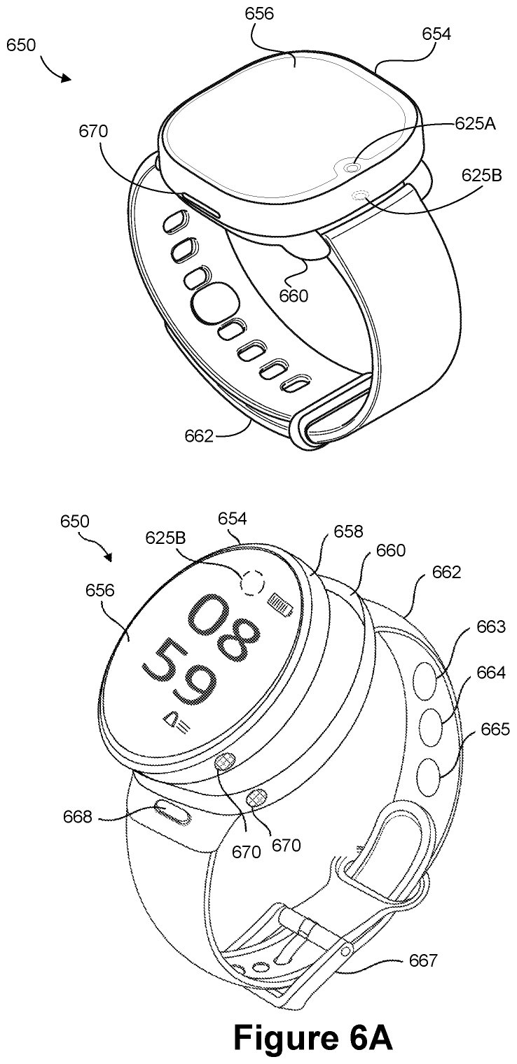

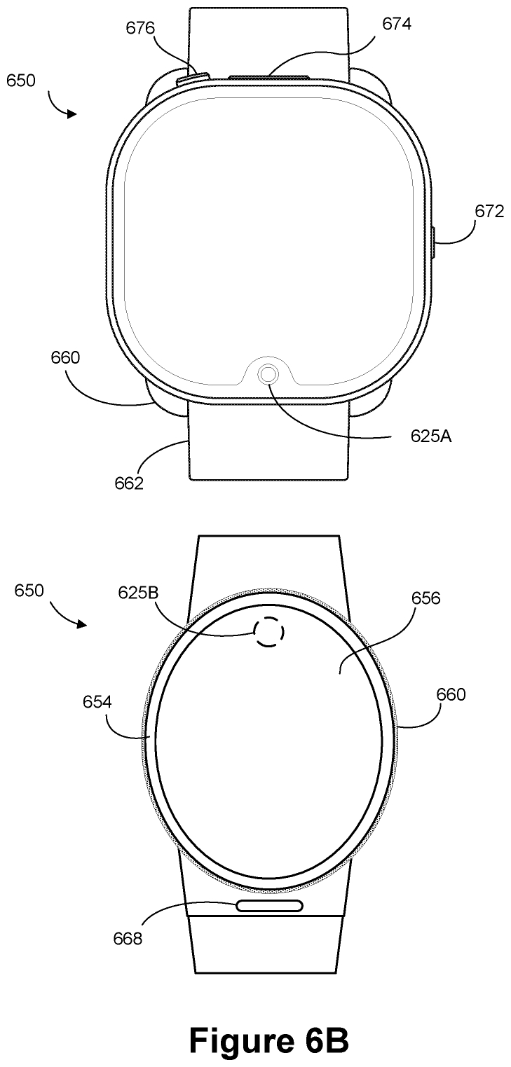

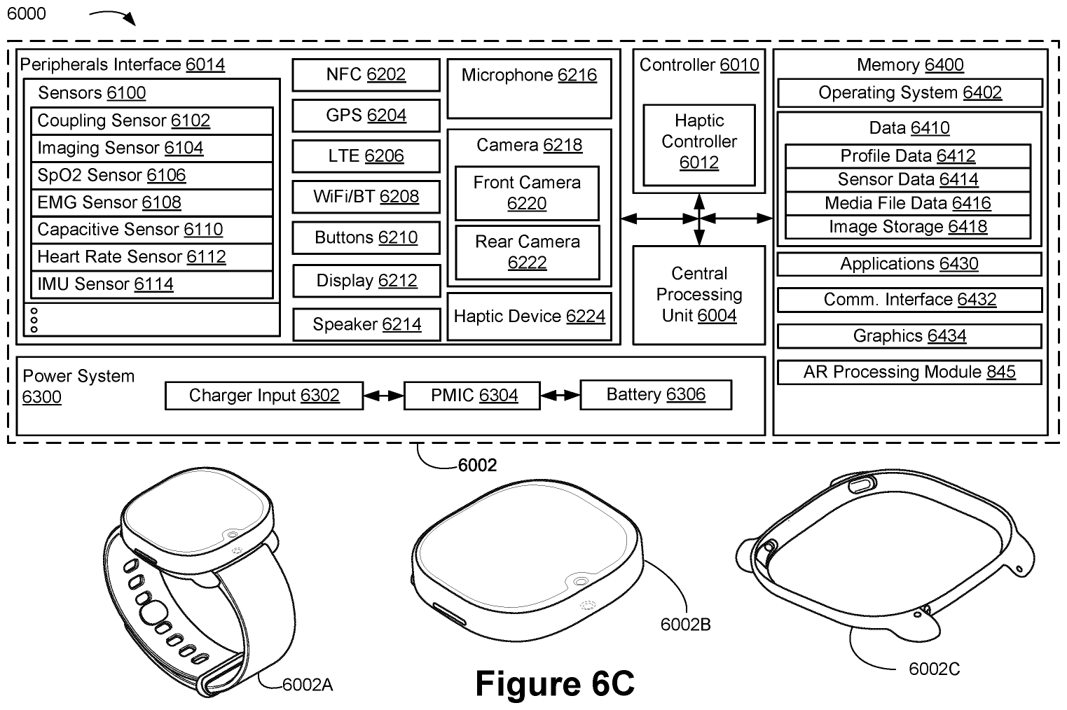

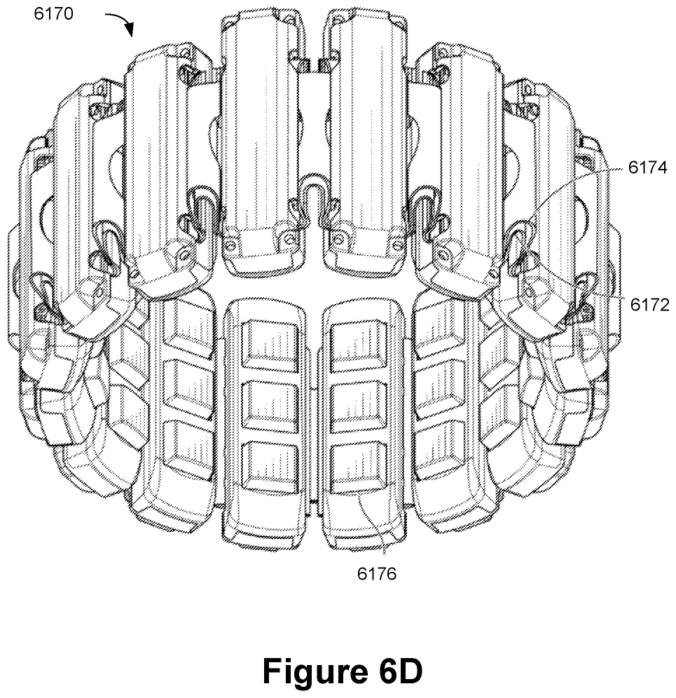

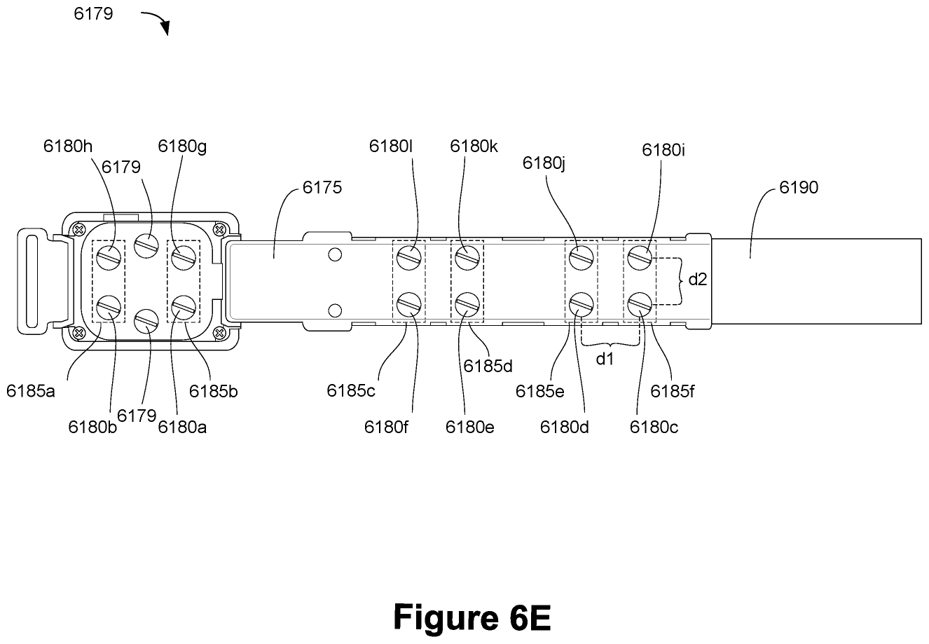

FIGS. 6A and 6B illustrate an example wrist-wearable device 650, in accordance with some embodiments. The wrist-wearable device 650 is an instance of the wearable device described herein (e.g., wrist-wearable device 120), such that the wearable device should be understood to have the features of the wrist-wearable device 650 and vice versa. FIG. 6A illustrates a perspective view of the wrist-wearable device 650 that includes a watch body 654 coupled with a watch band 662. The watch body 654 and the watch band 662 can have a substantially rectangular or circular shape and can be configured to allow a user to wear the wrist-wearable device 650 on a body part (e.g., a wrist). The wrist-wearable device 650 can include a retaining mechanism 667 (e.g., a buckle, a hook and loop fastener, etc.) for securing the watch band 662 to the user's wrist. The wrist-wearable device 650 can also include a coupling mechanism 660 (e.g., a cradle) for detachably coupling the capsule or watch body 654 (via a coupling surface of the watch body 654) to the watch band 962.

The wrist-wearable device 650 can perform various functions associated with navigating through user interfaces and selectively opening applications, as described above with reference to FIGS. 1A-5. As will be described in more detail below, operations executed by the wrist-wearable device 650 can include, without limitation, display of visual content to the user (e.g., visual content displayed on display 656); sensing user input (e.g., sensing a touch on peripheral button 668, sensing biometric data on sensor 664, sensing neuromuscular signals on neuromuscular sensor 665, etc.); messaging (e.g., text, speech, video, etc.); image capture; wireless communications (e.g., cellular, near field, Wi-Fi, personal area network, etc.); location determination; financial transactions; providing haptic feedback; alarms; notifications; biometric authentication; health monitoring; sleep monitoring; etc. These functions can be executed independently in the watch body 654, independently in the watch band 662, and/or in communication between the watch body 654 and the watch band 662. In some embodiments, functions can be executed on the wrist-wearable device 650 in conjunction with an artificial-reality environment that includes, but is not limited to, virtual-reality (VR) environments (including non-immersive, semi-immersive, and fully immersive VR environments); augmented-reality environments (including marker-based augmented-reality environments, markerless augmented-reality environments, location-based augmented-reality environments, and projection-based augmented-reality environments); hybrid reality; and other types of mixed-reality environments. As the skilled artisan will appreciate upon reading the descriptions provided herein, the novel wearable devices described herein can be used with any of these types of artificial-reality environments.

The watch band 662 can be configured to be worn by a user such that an inner surface of the watch band 662 is in contact with the user's skin. When worn by a user, sensor 664 is in contact with the user's skin. The sensor 664 can be a biosensor that senses a user's heart rate, saturated oxygen level, temperature, sweat level, muscle intentions, or a combination thereof. The watch band 662 can include multiple sensors 664 that can be distributed on an inside and/or an outside surface of the watch band 662. Additionally, or alternatively, the watch body 654 can include sensors that are the same or different than those of the watch band 662 (or the watch band 662 can include no sensors at all in some embodiments). For example, multiple sensors can be distributed on an inside and/or an outside surface of the watch body 654. As described below with reference to FIGS. 6B and/or 6C, the watch body 654 can include, without limitation, a front-facing image sensor 625A and/or a rear-facing image sensor 625B, a biometric sensor, an IMU, a heart rate sensor, a saturated oxygen sensor, a neuromuscular sensor(s), an altimeter sensor, a temperature sensor, a bioimpedance sensor, a pedometer sensor, an optical sensor (e.g., imaging sensor 6104), a touch sensor, a sweat sensor, etc. The sensor 664 can also include a sensor that provides data about a user's environment including a user's motion (e.g., an IMU), altitude, location, orientation, gait, or a combination thereof. The sensor 664 can also include a light sensor (e.g., an infrared light sensor, a visible light sensor) that is configured to track a position and/or motion of the watch body 654 and/or the watch band 662. The watch band 662 can transmit the data acquired by sensor 664 to the watch body 654 using a wired communication method (e.g., a Universal Asynchronous Receiver/Transmitter (UART), a USB transceiver, etc.) and/or a wireless communication method (e.g., near field communication, Bluetooth, etc.). The watch band 662 can be configured to operate (e.g., to collect data using sensor 664) independent of whether the watch body 654 is coupled to or decoupled from watch band 662.

In some examples, the watch band 662 can include a neuromuscular sensor 665 (e.g., an EMG sensor, a mechanomyogram (MMG) sensor, a sonomyography (SMG) sensor, etc.). Neuromuscular sensor 665 can sense a user's intention to perform certain motor actions. The sensed muscle intention can be used to control certain user interfaces displayed on the display 656 of the wrist-wearable device 650 and/or can be transmitted to a device responsible for rendering an artificial-reality environment (e.g., a head-mounted display) to perform an action in an associated artificial-reality environment, such as to control the motion of a virtual device displayed to the user.

Signals from neuromuscular sensor 665 can be used to provide a user with an enhanced interaction with a physical object and/or a virtual object in an artificial-reality application generated by an artificial-reality system (e.g., user interface objects presented on the display 656, or another computing device (e.g., a smartphone)). Signals from neuromuscular sensor 665 can be obtained (e.g., sensed and recorded) by one or more neuromuscular sensors 665 of the watch band 662. Although FIG. 6A shows one neuromuscular sensor 665, the watch band 662 can include a plurality of neuromuscular sensors 665 arranged circumferentially on an inside surface of the watch band 662 such that the plurality of neuromuscular sensors 665 contact the skin of the user. The watch band 662 can include a plurality of neuromuscular sensors 665 arranged circumferentially on an inside surface of the watch band 662. Neuromuscular sensor 665 can sense and record neuromuscular signals from the user as the user performs muscular activations (e.g., movements, gestures, etc.). The muscular activations performed by the user can include static gestures, such as placing the user's hand palm down on a table; dynamic gestures, such as grasping a physical or virtual object; and covert gestures that are imperceptible to another person, such as slightly tensing a joint by co-contracting opposing muscles or using sub-muscular activations. The muscular activations performed by the user can include symbolic gestures (e.g., gestures mapped to other gestures, interactions, or commands, for example, based on a gesture vocabulary that specifies the mapping of gestures to commands).

The watch band 662 and/or watch body 654 can include a haptic device 663 (e.g., a vibratory haptic actuator) that is configured to provide haptic feedback (e.g., a cutaneous and/or kinesthetic sensation, etc.) to the user's skin. The sensors 664 and 665, and/or the haptic device 663 can be configured to operate in conjunction with multiple applications including, without limitation, health monitoring, social media, game playing, and artificial reality (e.g., the applications associated with artificial reality).

The wrist-wearable device 650 can include a coupling mechanism (also referred to as a cradle) for detachably coupling the watch body 654 to the watch band 662. A user can detach the watch body 654 from the watch band 662 in order to reduce the encumbrance of the wrist-wearable device 650 to the user. The wrist-wearable device 650 can include a coupling surface on the watch body 654 and/or coupling mechanism(s) 660 (e.g., a cradle, a tracker band, a support base, a clasp). A user can perform any type of motion to couple the watch body 654 to the watch band 662 and to decouple the watch body 654 from the watch band 662. For example, a user can twist, slide, turn, push, pull, or rotate the watch body 654 relative to the watch band 662, or a combination thereof, to attach the watch body 654 to the watch band 662 and to detach the watch body 654 from the watch band 662.

As shown in the example of FIG. 6A, the watch band coupling mechanism 660 can include a type of frame or shell that allows the watch body 654 coupling surface to be retained within the watch band coupling mechanism 660. The watch body 654 can be detachably coupled to the watch band 662 through a friction fit, magnetic coupling, a rotation-based connector, a shear-pin coupler, a retention spring, one or more magnets, a clip, a pin shaft, a hook and loop fastener, or a combination thereof. In some examples, the watch body 654 can be decoupled from the watch band 662 by actuation of the release mechanism 670. The release mechanism 670 can include, without limitation, a button, a knob, a plunger, a handle, a lever, a fastener, a clasp, a dial, a latch, or a combination thereof.

As shown in FIGS. 6A-6B, the coupling mechanism 660 can be configured to receive a coupling surface proximate to the bottom side of the watch body 654 (e.g., a side opposite to a front side of the watch body 654 where the display 656 is located), such that a user can push the watch body 654 downward into the coupling mechanism 660 to attach the watch body 654 to the coupling mechanism 660. In some embodiments, the coupling mechanism 660 can be configured to receive a top side of the watch body 654 (e.g., a side proximate to the front side of the watch body 654 where the display 656 is located) that is pushed upward into the cradle, as opposed to being pushed downward into the coupling mechanism 660. In some embodiments, the coupling mechanism 660 is an integrated component of the watch band 662 such that the watch band 662 and the coupling mechanism 660 are a single unitary structure.

The wrist-wearable device 650 can include a single release mechanism 670 or multiple release mechanisms 670 (e.g., two release mechanisms 670 positioned on opposing sides of the wrist-wearable device 650 such as spring-loaded buttons). As shown in FIG. 6A, the release mechanism 670 can be positioned on the watch body 654 and/or the watch band coupling mechanism 660. Although FIG. 6A shows release mechanism 670 positioned at a corner of watch body 654 and at a corner of watch band coupling mechanism 660, the release mechanism 670 can be positioned anywhere on watch body 654 and/or watch band coupling mechanism 660 that is convenient for a user of wrist-wearable device 650 to actuate. A user of the wrist-wearable device 650 can actuate the release mechanism 670 by pushing, turning, lifting, depressing, shifting, or performing other actions on the release mechanism 670. Actuation of the release mechanism 670 can release (e.g., decouple) the watch body 654 from the watch band coupling mechanism 660 and the watch band 662 allowing the user to use the watch body 654 independently from watch band 662. For example, decoupling the watch body 654 from the watch band 662 can allow the user to capture images using rear-facing image sensor 625B.

FIG. 6B includes top views of examples of the wrist-wearable device 650. The examples of the wrist-wearable device 650 shown in FIGS. 6A-6B can include a coupling mechanism 660 (as shown in FIG. 6B, the shape of the coupling mechanism can correspond to the shape of the watch body 654 of the wrist-wearable device 650). The watch body 654 can be detachably coupled to the coupling mechanism 660 through a friction fit, magnetic coupling, a rotation-based connector, a shear-pin coupler, a retention spring, one or more magnets, a clip, a pin shaft, a hook and loop fastener, or any combination thereof.

In some examples, the watch body 654 can be decoupled from the coupling mechanism 660 by actuation of a release mechanism 670. The release mechanism 670 can include, without limitation, a button, a knob, a plunger, a handle, a lever, a fastener, a clasp, a dial, a latch, or a combination thereof. In some examples, the wristband system functions can be executed independently in the watch body 654, independently in the coupling mechanism 660, and/or in communication between the watch body 654 and the coupling mechanism 660. The coupling mechanism 660 can be configured to operate independently (e.g., execute functions independently) from watch body 654. Additionally, or alternatively, the watch body 654 can be configured to operate independently (e.g., execute functions independently) from the coupling mechanism 660. As described below with reference to the block diagram of FIG. 6A, the coupling mechanism 660 and/or the watch body 654 can each include the independent resources required to independently execute functions. For example, the coupling mechanism 660 and/or the watch body 654 can each include a power source (e.g., a battery), a memory, data storage, a processor (e.g., a central processing unit (CPU)), communications, a light source, and/or input/output devices.

The wrist-wearable device 650 can have various peripheral buttons 672, 674, and 676, for performing various operations at the wrist-wearable device 650. Also, various sensors, including one or both of the sensors 664 and 665, can be located on the bottom of the watch body 654, and can optionally be used even when the watch body 654 is detached from the watch band 662.

FIG. 6C is a block diagram of a computing system 6000, according to at least one embodiment of the present disclosure. The computing system 6000 includes an electronic device 6002, which can be, for example, a wrist-wearable device. The wrist-wearable device 650 described in detail above with respect to FIGS. 6A-6B is an example of the electronic device 6002, so the electronic device 6002 will be understood to include the components shown and described below for the computing system 6000. In some embodiments, all, or a substantial portion of the components of the computing system 6000 are included in a single integrated circuit. In some embodiments, the computing system 6000 can have a split architecture (e.g., a split mechanical architecture, a split electrical architecture) between a watch body (e.g., a watch body 654 in FIGS. 6A-6B) and a watch band (e.g., a watch band 662 in FIGS. 6A-6B). The electronic device 6002 can include a processor (e.g., a central processing unit 6004), a controller 6010, a peripherals interface 6014 that includes one or more sensors 6100 and various peripheral devices, a power source (e.g., a power system 6300), and memory (e.g., a memory 6400) that includes an operating system (e.g., an operating system 6402), data (e.g., data 6410), and one or more applications (e.g., applications 6430).

In some embodiments, the computing system 6000 includes the power system 6300 which includes a charger input 6302, a power-management integrated circuit (PMIC) 6304, and a battery 6306.

In some embodiments, a watch body and a watch band can each be electronic devices 6002 that each have respective batteries (e.g., battery 6306), and can share power with each other. The watch body and the watch band can receive a charge using a variety of techniques. In some embodiments, the watch body and the watch band can use a wired charging assembly (e.g., power cords) to receive the charge. Alternatively, or in addition, the watch body and/or the watch band can be configured for wireless charging. For example, a portable charging device can be designed to mate with a portion of watch body and/or watch band and wirelessly deliver usable power to a battery of watch body and/or watch band.

The watch body and the watch band can have independent power systems 6300 to enable each to operate independently. The watch body and watch band can also share power (e.g., one can charge the other) via respective PMICs 6304 that can share power over power and ground conductors and/or over wireless charging antennas.

In some embodiments, the peripherals interface 6014 can include one or more sensors 6100. The sensors 6100 can include a coupling sensor 6102 for detecting when the electronic device 6002 is coupled with another electronic device 6002 (e.g., a watch body can detect when it is coupled to a watch band, and vice versa). The sensors 6100 can include imaging sensors 6104 for collecting imaging data, which can optionally be the same device as one or more of the cameras 6218. In some embodiments, the imaging sensors 6104 can be separate from the cameras 6218. In some embodiments the sensors include an SpO2 sensor 6106. In some embodiments, the sensors 6100 include an EMG sensor 6108 for detecting, for example muscular movements by a user of the electronic device 6002. In some embodiments, the sensors 6100 include a capacitive sensor 6110 for detecting changes in potential of a portion of a user's body. In some embodiments, the sensors 6100 include a heart rate sensor 6112. In some embodiments, the sensors 6100 include an inertial measurement unit (IMU) sensor 6114 for detecting, for example, changes in acceleration of the user's hand.

In some embodiments, the peripherals interface 6014 includes a near-field communication (NFC) component 6202, a global-position system (GPS) component 6204, a long-term evolution (LTE) component 6206, and or a Wi-Fi or Bluetooth communication component 6208.

In some embodiments, the peripherals interface includes one or more buttons (e.g., the peripheral buttons 672, 674, and 676 in FIG. 6B), which, when selected by a user, cause operation to be performed at the electronic device 6002.

The electronic device 6002 can include at least one display 6212, for displaying visual affordances to the user, including user-interface elements and/or three-dimensional virtual objects. The display can also include a touch screen for inputting user inputs, such as touch gestures, swipe gestures, and the like.

The electronic device 6002 can include at least one speaker 6214 and at least one microphone 6216 for providing audio signals to the user and receiving audio input from the user. The user can provide user inputs through the microphone 6216 and can also receive audio output from the speaker 6214 as part of a haptic event provided by the haptic controller 6012.

The electronic device 6002 can include at least one camera 6218, including a front camera 6220 and a rear camera 6222. In some embodiments, the electronic device 6002 can be a head-wearable device, and one of the cameras 6218 can be integrated with a lens assembly of the head-wearable device.

One or more of the electronic devices 6002 can include one or more haptic controllers 6012 and associated componentry for providing haptic events at one or more of the electronic devices 6002 (e.g., a vibrating sensation or audio output in response to an event at the electronic device 6002). The haptic controllers 6012 can communicate with one or more electroacoustic devices, including a speaker of the one or more speakers 6214 and/or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). The haptic controller 6012 can provide haptic events to that are capable of being sensed by a user of the electronic devices 6002. In some embodiments, the one or more haptic controllers 6012 can receive input signals from an application of the applications 6430.

Memory 6400 optionally includes high-speed random-access memory and optionally also includes non-volatile memory, such as one or more magnetic disk storage devices, flash memory devices, or other non-volatile solid-state memory devices. Access to the memory 6400 by other components of the electronic device 6002, such as the one or more processors of the central processing unit 6004, and the peripherals interface 6014 is optionally controlled by a memory controller of the controllers 6010.

In some embodiments, software components stored in the memory 6400 can include one or more operating systems 6402 (e.g., a Linux-based operating system, an Android operating system, etc.). The memory 6400 can also include data 6410, including structured data (e.g., SQL databases, MongoDB databases, GraphQL data, JSON data, etc.). The data 6410 can include profile data 6412, sensor data 6414, media file data 6416, and image storage 6418.

In some embodiments, software components stored in the memory 6400 include one or more applications 6430 configured to be perform operations at the electronic devices 6002. In some embodiments, the software components stored in the memory 6400 one or more communication interface modules 6432, one or more graphics modules 6434, and an AR processing module 845 (FIGS. 8A and 8B). In some embodiments, a plurality of applications 6430 and modules can work in conjunction with one another to perform various tasks at one or more of the electronic devices 6002.

In some embodiments, software components stored in the memory 6400 include one or more applications 6430 configured to be perform operations at the electronic devices 6002. In some embodiments, the one or more applications 6430 include one or more communication interface modules 6432, one or more graphics modules 6434, one or more camera application modules 6436. In some embodiments, a plurality of applications 6430 can work in conjunction with one another to perform various tasks at one or more of the electronic devices 6002.

It should be appreciated that the electronic devices 6002 are only some examples of the electronic devices 6002 within the computing system 6000, and that other electronic devices 6002 that are part of the computing system 6000 can have more or fewer components than shown optionally combines two or more components, or optionally have a different configuration or arrangement of the components. The various components shown in FIG. 6C are implemented in hardware, software, firmware, or a combination thereof, including one or more signal processing and/or application-specific integrated circuits.

As illustrated by the lower portion of FIG. 6C, various individual components of a wrist-wearable device can be examples of the electronic device 6002. For example, some or all of the components shown in the electronic device 6002 can be housed or otherwise disposed in a combined watch device 6002A, or within individual components of the capsule device watch body 6002B, the cradle portion 6002C, and/or a watch band.

FIG. 6D illustrates a wearable device 6170, in accordance with some embodiments. In some embodiments, the wearable device 6170 is used to generate control information (e.g., sensed data about neuromuscular signals or instructions to perform certain commands after the data is sensed) for causing a computing device to perform one or more input commands. In some embodiments, the wearable device 6170 includes a plurality of neuromuscular sensors 6176. In some embodiments, the plurality of neuromuscular sensors 6176 includes a predetermined number of (e.g., 16) neuromuscular sensors (e.g., EMG sensors) arranged circumferentially around an elastic band 6174. The plurality of neuromuscular sensors 6176 may include any suitable number of neuromuscular sensors. In some embodiments, the number and arrangement of neuromuscular sensors 6176 depends on the particular application for which the wearable device 6170 is used. For instance, a wearable device 6170 configured as an armband, wristband, or chest-band may include a plurality of neuromuscular sensors 6176 with different number of neuromuscular sensors and different arrangement for each use case, such as medical use cases as compared to gaming or general day-to-day use cases. For example, at least 16 neuromuscular sensors 6176 may be arranged circumferentially around elastic band 6174.

In some embodiments, the elastic band 6174 is configured to be worn around a user's lower arm or wrist. The elastic band 6174 may include a flexible electronic connector 6172. In some embodiments, the flexible electronic connector 6172 interconnects separate sensors and electronic circuitry that are enclosed in one or more sensor housings. Alternatively, in some embodiments, the flexible electronic connector 6172 interconnects separate sensors and electronic circuitry that are outside of the one or more sensor housings. Each neuromuscular sensor of the plurality of neuromuscular sensors 6176 can include a skin-contacting surface that includes one or more electrodes. One or more sensors of the plurality of neuromuscular sensors 6176 can be coupled together using flexible electronics incorporated into the wearable device 6170. In some embodiments, one or more sensors of the plurality of neuromuscular sensors 6176 can be integrated into a woven fabric, wherein the fabric one or more sensors of the plurality of neuromuscular sensors 6176 are sewn into the fabric and mimic the pliability of fabric (e.g., the one or more sensors of the plurality of neuromuscular sensors 6176 can be constructed from a series woven strands of fabric). In some embodiments, the sensors are flush with the surface of the textile and are indistinguishable from the textile when worn by the user.

FIG. 6E illustrates a wearable device 6179 in accordance with some embodiments. The wearable device 6179 includes paired sensor channels 6185a-6185f along an interior surface of a wearable structure 6175 that are configured to detect neuromuscular signals. Different number of paired sensors channels can be used (e.g., one pair of sensors, three pairs of sensors, four pairs of sensors, or six pairs of sensors). The wearable structure 6175 can include a band portion 6190, a capsule portion 6195, and a cradle portion (not pictured) that is coupled with the band portion 6190 to allow for the capsule portion 6195 to be removably coupled with the band portion 6190. For embodiments in which the capsule portion 6195 is removable, the capsule portion 6195 can be referred to as a removable structure, such that in these embodiments the wearable device includes a wearable portion (e.g., band portion 6190 and the cradle portion) and a removable structure (the removable capsule portion which can be removed from the cradle). In some embodiments, the capsule portion 6195 includes the one or more processors and/or other components of the wearable device 888 described above in reference to FIGS. 8A and 8B. The wearable structure 6175 is configured to be worn by a user 115. More specifically, the wearable structure 6175 is configured to couple the wearable device 6179 to a wrist, arm, forearm, or other portion of the user's body. Each paired sensor channels 6185a-6185f includes two electrodes 6180 (e.g., electrodes 6180a-6180h) for sensing neuromuscular signals based on differential sensing within each respective sensor channel. In accordance with some embodiments, the wearable device 6170 further includes an electrical ground and a shielding electrode.

The techniques described above can be used with any device for sensing neuromuscular signals, including the arm-wearable devices of FIG. 6A-6C, but could also be used with other types of wearable devices for sensing neuromuscular signals (such as body-wearable or head-wearable devices that might have neuromuscular sensors closer to the brain or spinal column).

In some embodiments, a wrist-wearable device can be used in conjunction with a head-wearable device described below, and the wrist-wearable device can also be configured to be used to allow a user to control aspect of the artificial reality (e.g., by using EMG-based gestures to control user interface objects in the artificial reality and/or by allowing a user to interact with the touchscreen on the wrist-wearable device to also control aspects of the artificial reality). Having thus described example wrist-wearable device, attention will now be turned to example head-wearable devices, such AR glasses and VR headsets.

### Example Head-Wearable Devices

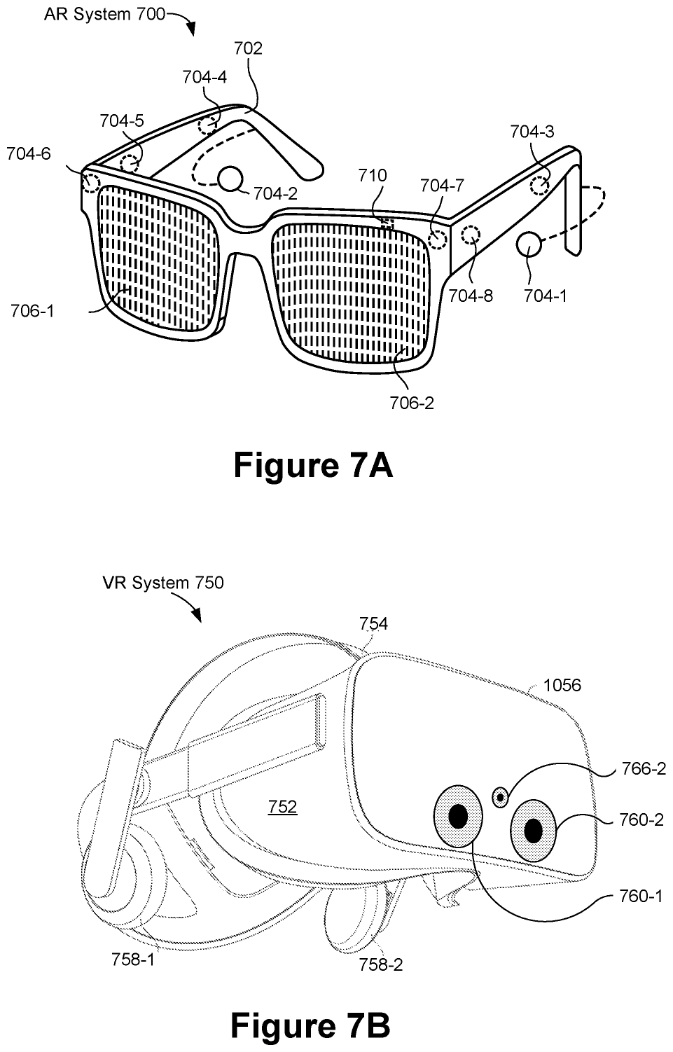

FIG. 7A shows an example AR system 700 in accordance with some embodiments. In FIG. 7A, the AR system 700 includes an eyewear device with a frame 702 configured to hold a left display device 706-1 and a right display device 706-2 in front of a user's eyes. The display devices 706-1 and 706-2 may act together or independently to present an image or series of images to a user. While the AR system 700 includes two displays, embodiments of this disclosure may be implemented in AR systems with a single near-eye display (NED) or more than two NEDs.

In some embodiments, the AR system 700 includes one or more sensors, such as the acoustic sensors 704. For example, the acoustic sensors 704 can generate measurement signals in response to motion of the AR system 700 and may be located on substantially any portion of the frame 702. Any one of the sensors may be a position sensor, an IMU, a depth camera assembly, or any combination thereof. In some embodiments, the AR system 700 includes more or fewer sensors than are shown in FIG. 7A. In embodiments in which the sensors include an IMU, the IMU may generate calibration data based on measurement signals from the sensors. Examples of the sensors include, without limitation, accelerometers, gyroscopes, magnetometers, other suitable types of sensors that detect motion, sensors used for error correction of the IMU, or some combination thereof.

In some embodiments, the AR system 700 includes a microphone array with a plurality of acoustic sensors 704-1 through 704-8, referred to collectively as the acoustic sensors 704. The acoustic sensors 704 may be transducers that detect air pressure variations induced by sound waves. In some embodiments, each acoustic sensor 704 is configured to detect sound and convert the detected sound into an electronic format (e.g., an analog or digital format). In some embodiments, the microphone array includes ten acoustic sensors: 704-1 and 704-2 designed to be placed inside a corresponding ear of the user, acoustic sensors 704-3, 704-4, 704-5, 704-6, 704-7, and 704-8 positioned at various locations on the frame 702, and acoustic sensors positioned on a corresponding neckband, where the neckband is an optional component of the system that is not present in certain embodiments of the artificial-reality systems discussed herein.

The configuration of the acoustic sensors 704 of the microphone array may vary. While the AR system 700 is shown in FIG. 7A having ten acoustic sensors 704, the number of acoustic sensors 704 may be more or fewer than ten. In some situations, using more acoustic sensors 704 increases the amount of audio information collected and/or the sensitivity and accuracy of the audio information. In contrast, in some situations, using a lower number of acoustic sensors 704 decreases the computing power required by a controller to process the collected audio information. In addition, the position of each acoustic sensor 704 of the microphone array may vary. For example, the position of an acoustic sensor 704 may include a defined position on the user, a defined coordinate on the frame 702, an orientation associated with each acoustic sensor, or some combination thereof.

The acoustic sensors 704-1 and 704-2 may be positioned on different parts of the user's ear. In some embodiments, there are additional acoustic sensors on or surrounding the ear in addition to acoustic sensors 704 inside the ear canal. In some situations, having an acoustic sensor positioned next to an ear canal of a user enables the microphone array to collect information on how sounds arrive at the ear canal. By positioning at least two of the acoustic sensors 704 on either side of a user's head (e.g., as binaural microphones), the AR device 700 is able to simulate binaural hearing and capture a 3D stereo sound field around a user's head. In some embodiments, the acoustic sensors 704-1 and 704-2 are connected to the AR system 700 via a wired connection, and in other embodiments, the acoustic sensors 704-1 and 704-2 are connected to the AR system 700 via a wireless connection (e.g., a Bluetooth connection). In some embodiments, the AR system 700 does not include the acoustic sensors 704-1 and 704-2.

The acoustic sensors 704 on the frame 702 may be positioned along the length of the temples, across the bridge of the nose, above or below the display devices 706, or in some combination thereof. The acoustic sensors 704 may be oriented such that the microphone array is able to detect sounds in a wide range of directions surrounding the user that is wearing the AR system 700. In some embodiments, a calibration process is performed during manufacturing of the AR system 700 to determine relative positioning of each acoustic sensor 704 in the microphone array.

In some embodiments, the eyewear device further includes, or is communicatively coupled to, an external device (e.g., a paired device), such as the optional neckband discussed above. In some embodiments, the optional neckband is coupled to the eyewear device via one or more connectors. The connectors may be wired or wireless connectors and may include electrical and/or non-electrical (e.g., structural) components. In some embodiments, the eyewear device and the neckband operate independently without any wired or wireless connection between them. In some embodiments, the components of the eyewear device and the neckband are located on one or more additional peripheral devices paired with the eyewear device, the neckband, or some combination thereof. Furthermore, the neckband is intended to represent any suitable type or form of paired device. Thus, the following discussion of neckband may also apply to various other paired devices, such as smart watches, smart phones, wrist bands, other wearable devices, hand-held controllers, tablet computers, or laptop computers.

In some situations, pairing external devices, such as the optional neckband, with the AR eyewear device enables the AR eyewear device to achieve the form factor of a pair of glasses while still providing sufficient battery and computation power for expanded capabilities. Some, or all, of the battery power, computational resources, and/or additional features of the AR system 700 may be provided by a paired device or shared between a paired device and an eyewear device, thus reducing the weight, heat profile, and form factor of the eyewear device overall while still retaining desired functionality. For example, the neckband may allow components that would otherwise be included on an eyewear device to be included in the neckband thereby shifting a weight load from a user's head to a user's shoulders. In some embodiments, the neckband has a larger surface area over which to diffuse and disperse heat to the ambient environment. Thus, the neckband may allow for greater battery and computation capacity than might otherwise have been possible on a stand-alone eyewear device. Because weight carried in the neckband may be less invasive to a user than weight carried in the eyewear device, a user may tolerate wearing a lighter eyewear device and carrying or wearing the paired device for greater lengths of time than the user would tolerate wearing a heavy, stand-alone eyewear device, thereby enabling an artificial-reality environment to be incorporated more fully into a user's day-to-day activities.

In some embodiments, the optional neckband is communicatively coupled with the eyewear device and/or to other devices. The other devices may provide certain functions (e.g., tracking, localizing, depth mapping, processing, storage, etc.) to the AR system 700. In some embodiments, the neckband includes a controller and a power source. In some embodiments, the acoustic sensors of the neckband are configured to detect sound and convert the detected sound into an electronic format (analog or digital).

The controller of the neckband processes information generated by the sensors on the neckband and/or the AR system 700. For example, the controller may process information from the acoustic sensors 704. For each detected sound, the controller may perform a direction of arrival (DOA) estimation to estimate a direction from which the detected sound arrived at the microphone array. As the microphone array detects sounds, the controller may populate an audio data set with the information. In embodiments in which the AR system 700 includes an IMU, the controller may compute all inertial and spatial calculations from the IMU located on the eyewear device. The connector may convey information between the eyewear device and the neckband and between the eyewear device and the controller. The information may be in the form of optical data, electrical data, wireless data, or any other transmittable data form. Moving the processing of information generated by the eyewear device to the neckband may reduce weight and heat in the eyewear device, making it more comfortable and safer for a user.

In some embodiments, the power source in the neckband provides power to the eyewear device and the neckband. The power source may include, without limitation, lithium-ion batteries, lithium-polymer batteries, primary lithium batteries, alkaline batteries, or any other form of power storage. In some embodiments, the power source is a wired power source.

As noted, some artificial-reality systems may, instead of blending an artificial reality with actual reality, substantially replace one or more of a user's sensory perceptions of the real world with a virtual experience. One example of this type of system is a head-worn display system, such as the VR system 750 in FIG. 7B, which mostly or completely covers a user's field of view.

FIG. 7B shows a VR system 750 (e.g., also referred to herein as VR headsets or VR headset) in accordance with some embodiments. The VR system 750 includes a head-mounted display (HMD) 752. The HMD 752 includes a front body 756 and a frame 754 (e.g., a strap or band) shaped to fit around a user's head. In some embodiments, the HMD 752 includes output audio transducers 758-1 and 758-2, as shown in FIG. 7B (e.g., transducers). In some embodiments, the front body 756 and/or the frame 754 includes one or more electronic elements, including one or more electronic displays, one or more IMUs, one or more tracking emitters or detectors, and/or any other suitable device or sensor for creating an artificial-reality experience.

Artificial-reality systems may include a variety of types of visual feedback mechanisms. For example, display devices in the AR system 700 and/or the VR system 750 may include one or more liquid-crystal displays (LCDs), light emitting diode (LED) displays, organic LED (OLED) displays, and/or any other suitable type of display screen. Artificial-reality systems may include a single display screen for both eyes or may provide a display screen for each eye, which may allow for additional flexibility for varifocal adjustments or for correcting a refractive error associated with the user's vision. Some artificial-reality systems also include optical subsystems having one or more lenses (e.g., conventional concave or convex lenses, Fresnel lenses, or adjustable liquid lenses) through which a user may view a display screen.

In addition to or instead of using display screens, some artificial-reality systems include one or more projection systems. For example, display devices in the AR system 700 and/or the VR system 750 may include micro-LED projectors that project light (e.g., using a waveguide) into display devices, such as clear combiner lenses that allow ambient light to pass through. The display devices may refract the projected light toward a user's pupil and may enable a user to simultaneously view both artificial-reality content and the real world. Artificial-reality systems may also be configured with any other suitable type or form of image projection system.

Artificial-reality systems may also include various types of computer vision components and subsystems. For example, the AR system 700 and/or the VR system 750 can include one or more optical sensors such as two-dimensional (2D) or three-dimensional (3D) cameras, time-of-flight depth sensors, single-beam or sweeping laser rangefinders, 3D LiDAR sensors, and/or any other suitable type or form of optical sensor. An artificial-reality system may process data from one or more of these sensors to identify a location of a user, to map the real world, to provide a user with context about real-world surroundings, and/or to perform a variety of other functions. For example, FIG. 10B shows VR system 750 having cameras 760-1 and 760-2 that can be used to provide depth information for creating a voxel field and a two-dimensional mesh to provide object information to the user to avoid collisions. FIG. 7B also shows that the VR system includes one or more additional cameras 762 that are configured to augment the cameras 760-1 and 760-2 by providing more information. For example, the additional cameras 762 can be used to supply color information that is not discerned by cameras 760-1 and 760-2. In some embodiments, cameras 760-1 and 760-2 and additional cameras 762 can include an optional IR cut filter configured to remove IR light from being received at the respective camera sensors.

In some embodiments, the AR system 700 and/or the VR system 750 can include haptic (tactile) feedback systems, which may be incorporated into headwear, gloves, body suits, handheld controllers, environmental devices (e.g., chairs or floormats), and/or any other type of device or system, such as the wearable devices discussed herein. The haptic feedback systems may provide various types of cutaneous feedback, including vibration, force, traction, shear, texture, and/or temperature. The haptic feedback systems may also provide various types of kinesthetic feedback, such as motion and compliance. The haptic feedback may be implemented using motors, piezoelectric actuators, fluidic systems, and/or a variety of other types of feedback mechanisms. The haptic feedback systems may be implemented independently of other artificial-reality devices, within other artificial-reality devices, and/or in conjunction with other artificial-reality devices.

The techniques described above can be used with any device for interacting with an artificial-reality environment, including the head-wearable devices of FIG. 7A-7B, but could also be used with other types of wearable devices for sensing neuromuscular signals (such as body-wearable or head-wearable devices that might have neuromuscular sensors closer to the brain or spinal column). The AR system 700 and/or the VR system 750 are instances of the head-wearable device 110 and the AR headset described herein, such that the head-wearable device 110 and the AR headset should be understood to have the features of the AR system 700 and/or the VR system 750 and vice versa. Having thus described example wrist-wearable device and head-wearable devices, attention will now be turned to example feedback systems that can be integrated into the devices described above or be a separate device.

### Example Systems

FIGS. 8A and 8B are block diagrams illustrating an example artificial-reality system in accordance with some embodiments. The system 800 includes one or more devices for facilitating an interactivity with an artificial-reality environment in accordance with some embodiments. For example, the head-wearable device 811 can present to the user 8015 with a user interface within the artificial-reality environment. As a non-limiting example, the system 800 includes one or more wearable devices, which can be used in conjunction with one or more computing devices. In some embodiments, the system 800 provides the functionality of a virtual-reality device, an augmented-reality device, a mixed-reality device, hybrid-reality device, or a combination thereof. In some embodiments, the system 800 provides the functionality of a user interface and/or one or more user applications (e.g., games, word processors, messaging applications, calendars, clocks, etc.).

The system 800 can include one or more of servers 870, electronic devices 874 (e.g., a computer, 874a, a smartphone 874b, a controller 874c, and/or other devices), head-wearable devices 811 (e.g., the head-wearable device 110, the AR system 700 or the VR system 750), and/or wrist-wearable devices 888 (e.g., the wrist-wearable devices 120). In some embodiments, the one or more of servers 870, electronic devices 874, head-wearable devices 811, and/or wrist-wearable devices 888 are communicatively coupled via a network 872. In some embodiments, the head-wearable device 811 is configured to cause one or more operations to be performed by a communicatively coupled wrist-wearable device 888, and/or the two devices can also both be connected to an intermediary device, such as a smartphone 874b, a controller 874c, a portable computing unit, or other device that provides instructions and data to and between the two devices. In some embodiments, the head-wearable device 811 is configured to cause one or more operations to be performed by multiple devices in conjunction with the wrist-wearable device 888. In some embodiments, instructions to cause the performance of one or more operations are controlled via an artificial-reality processing module 845. The artificial-reality processing module 845 can be implemented in one or more devices, such as the one or more of servers 870, electronic devices 874, head-wearable devices 811, and/or wrist-wearable devices 888. In some embodiments, the one or more devices perform operations of the artificial-reality processing module 845, using one or more respective processors, individually or in conjunction with at least one other device as described herein. In some embodiments, the system 800 includes other wearable devices not shown in FIG. 8A and FIG. 8B, such as rings, collars, anklets, gloves, and the like.

In some embodiments, the system 800 provides the functionality to control or provide commands to the one or more computing devices 874 based on a wearable device (e.g., head-wearable device 811 or wrist-wearable device 888) determining motor actions or intended motor actions of the user. A motor action is an intended motor action when before the user performs the motor action or before the user completes the motor action, the detected neuromuscular signals travelling through the neuromuscular pathways can be determined to be the motor action. Motor actions can be detected based on the detected neuromuscular signals, but can additionally (using a fusion of the various sensor inputs), or alternatively, be detected using other types of sensors (such as cameras focused on viewing hand movements and/or using data from an inertial measurement unit that can detect characteristic vibration sequences or other data types to correspond to particular in-air hand gestures). The one or more computing devices include one or more of a head-mounted display, smartphones, tablets, smart watches, laptops, computer systems, augmented reality systems, robots, vehicles, virtual avatars, user interfaces, a wrist-wearable device, and/or other electronic devices and/or control interfaces.

In some embodiments, the motor actions include digit movements, hand movements, wrist movements, arm movements, pinch gestures, index finger movements, middle finger movements, ring finger movements, little finger movements, thumb movements, hand clenches (or fists), waving motions, and/or other movements of the user's hand or arm.

In some embodiments, the user can define one or more gestures using the learning module. In some embodiments, the user can enter a training phase in which a user defined gesture is associated with one or more input commands that when provided to a computing device cause the computing device to perform an action. Similarly, the one or more input commands associated with the user-defined gesture can be used to cause a wearable device to perform one or more actions locally. The user-defined gesture, once trained, is stored in the memory 860. Similar to the motor actions, the one or more processors 850 can use the detected neuromuscular signals by the one or more sensors 825 to determine that a user-defined gesture was performed by the user.

The electronic devices 874 can also include a communication interface 815d, an interface 820d (e.g., including one or more displays, lights, speakers, and haptic generators), one or more sensors 825d, one or more applications 835d, an artificial-reality processing module 845d, one or more processors 850d, and memory 860d. The electronic devices 874 are configured to communicatively couple with the wrist-wearable device 888 and/or head-wearable device 811 (or other devices) using the communication interface 815d. In some embodiments, the electronic devices 874 are configured to communicatively couple with the wrist-wearable device 888 and/or head-wearable device 811 (or other devices) via an application programming interface (API). In some embodiments, the electronic devices 874 operate in conjunction with the wrist-wearable device 888 and/or the head-wearable device 811 to determine a hand gesture and cause the performance of an operation or action at a communicatively coupled device.

The server 870 includes a communication interface 815e, one or more applications 835e, an artificial-reality processing module 845e, one or more processors 850e, and memory 860e. In some embodiments, the server 870 is configured to receive sensor data from one or more devices, such as the head-wearable device 811, the wrist-wearable device 888, and/or electronic device 874, and use the received sensor data to identify a gesture or user input. The server 870 can generate instructions that cause the performance of operations and actions associated with a determined gesture or user input at communicatively coupled devices, such as the head-wearable device 811.

The wrist-wearable device 888 includes a communication interface 815a, an interface 820a (e.g., including one or more displays, lights, speakers, and haptic generators), one or more applications 835a, an artificial-reality processing module 845a, one or more processors 850a, and memory 860a (including sensor data 862a and AR processing data 864a). In some embodiments, the wrist-wearable device 888 includes one or more sensors 825a, one or more haptic generators 821a, one or more imaging devices 855a (e.g., a camera), microphones, and/or speakers. The wrist-wearable device 888 can operate alone or in conjunction with another device, such as the head-wearable device 811, to perform one or more operations, such as capturing camera data, presenting a representation of the image data at a coupled display, operating one or more applications 835, and/or allowing a user to participate in an AR environment.

The head-wearable device 811 includes smart glasses (e.g., the augmented-reality glasses), artificial reality headsets (e.g., VR/AR headsets), or other head worn device. In some embodiments, one or more components of the head-wearable device 811 are housed within a body of the HMD 814 (e.g., frames of smart glasses, a body of a AR headset, etc.). In some embodiments, one or more components of the head-wearable device 811 are stored within or coupled with lenses of the HMD 814. Alternatively or in addition, in some embodiments, one or more components of the head-wearable device 811 are housed within a modular housing 806. The head-wearable device 811 is configured to communicatively couple with other electronic device 874 and/or a server 870 using communication interface 815 as discussed above.

FIG. 8B describes additional details of the HMD 814 and modular housing 806 described above in reference to 8A, in accordance with some embodiments.

The HMD 814 includes a communication interface 815, a display 830, an AR processing module 845, one or more processors, and memory. In some embodiments, the HMD 814 includes one or more sensors 825, one or more haptic generators 821, one or more imaging devices 855 (e.g., a camera), microphones 813, speakers 817, and/or one or more applications 835. The HMD 814 operates in conjunction with the housing 806 to perform one or more operations of a head-wearable device 811, such as capturing camera data, presenting a representation of the image data at a coupled display, operating one or more applications 835, and/or allowing a user to participate in an AR environment.

The housing 806 include(s) a communication interface 815, circuitry 846, a power source 807 (e.g., a battery for powering one or more electronic components of the housing 806 and/or providing usable power to the HMD 814), one or more processors 850, and memory 860. In some embodiments, the housing 806 can include one or more supplemental components that add to the functionality of the HMD 814. For example, in some embodiments, the housing 806 can include one or more sensors 825, an AR processing module 845, one or more haptic generators 821, one or more imaging devices 855, one or more microphones 813, one or more speakers 817, etc. The housing 106 is configured to couple with the HMD 814 via the one or more retractable side straps. More specifically, the housing 806 is a modular portion of the head-wearable device 811 that can be removed from head-wearable device 811 and replaced with another housing (which includes more or less functionality). The modularity of the housing 806 allows a user to adjust the functionality of the head-wearable device 811 based on their needs.

In some embodiments, the communications interface 815 is configured to communicatively couple the housing 806 with the HMD 814, the server 870, and/or other electronic device 874 (e.g., the controller 874c, a tablet, a computer, etc.). The communication interface 815 is used to establish wired or wireless connections between the housing 806 and the other devices. In some embodiments, the communication interface 815 includes hardware capable of data communications using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, or MiWi), custom or standard wired protocols (e.g., Ethernet or HomePlug), and/or any other suitable communication protocol. In some embodiments, the housing 806 is configured to communicatively couple with the HMD 814 and/or other electronic device 874 via an application programming interface (API).

In some embodiments, the power source 807 is a battery. The power source 807 can be a primary or secondary battery source for the HMD 814. In some embodiments, the power source 807 provides useable power to the one or more electrical components of the housing 806 or the HMD 814. For example, the power source 807 can provide usable power to the sensors 821, the speakers 817, the HMD 814, and the microphone 813. In some embodiments, the power source 807 is a rechargeable battery. In some embodiments, the power source 807 is a modular battery that can be removed and replaced with a fully charged battery while it is charged separately.

The one or more sensors 825 can include heart rate sensors, neuromuscular-signal sensors (e.g., electromyography (EMG) sensors), SpO2 sensors, altimeters, thermal sensors or thermal couples, ambient light sensors, ambient noise sensors, and/or inertial measurement units (IMU)s. Additional non-limiting examples of the one or more sensors 825 include, e.g., infrared, pyroelectric, ultrasonic, microphone, laser, optical, Doppler, gyro, accelerometer, resonant LC sensors, capacitive sensors, acoustic sensors, and/or inductive sensors. In some embodiments, the one or more sensors 825 are configured to gather additional data about the user (e.g., an impedance of the user's body). Examples of sensor data output by these sensors includes body temperature data, infrared range-finder data, positional information, motion data, activity recognition data, silhouette detection and recognition data, gesture data, heart rate data, and other wearable device data (e.g., biometric readings and output, accelerometer data). The one or more sensors 825 can include location sensing devices (e.g., GPS) configured to provide location information. In some embodiment, the data measured or sensed by the one or more sensors 825 is stored in memory 860. In some embodiments, the housing 806 receives sensor data from communicatively coupled devices, such as the HMD 814, the server 870, and/or other electronic device 874. Alternatively, the housing 806 can provide sensors data to the HMD 814, the server 870, and/or other electronic device 874.

The one or more haptic generators 821 can include one or more actuators (e.g., eccentric rotating mass (ERM), linear resonant actuators (LRA), voice coil motor (VCM), piezo haptic actuator, thermoelectric devices, solenoid actuators, ultrasonic transducers or sensors, etc.). In some embodiments, the one or more haptic generators 821 are hydraulic, pneumatic, electric, and/or mechanical actuators. In some embodiments, the one or more haptic generators 821 are part of a surface of the housing 806 that can be used to generate a haptic response (e.g., a thermal change at the surface, a tightening or loosening of a band, increase or decrease in pressure, etc.). For example, the one or more haptic generators 825 can apply vibration stimulations, pressure stimulations, squeeze simulations, shear stimulations, temperature changes, or some combination thereof to the user. In addition, in some embodiments, the one or more haptic generators 821 include audio generating devices (e.g., speakers 817 and other sound transducers) and illuminating devices (e.g., light-emitting diodes (LED)s, screen displays, etc.). The one or more haptic generators 821 can be used to generate different audible sounds and/or visible lights that are provided to the user as haptic responses. The above list of haptic generators is non-exhaustive; any affective devices can be used to generate one or more haptic responses that are delivered to a user.

In some embodiments, the one or more applications 835 include social-media applications, banking applications, health applications, messaging applications, web browsers, gaming application, streaming applications, media applications, imaging applications, productivity applications, social applications, etc. In some embodiments, the one or more applications 835 include artificial reality applications. The one or more applications 835 are configured to provide data to the head-wearable device 811 for performing one or more operations. In some embodiments, the one or more applications 835 can be displayed via a display 830 of the head-wearable device 811 (e.g., via the HMD 814).

In some embodiments, instructions to cause the performance of one or more operations are controlled via AR processing module 845. The AR processing module 845 can be implemented in one or more devices, such as the one or more of servers 870, electronic devices 874, head-wearable devices 811, and/or wrist-wearable devices 870. In some embodiments, the one or more devices perform operations of the AR processing module 845, using one or more respective processors, individually or in conjunction with at least one other device as described herein. In some embodiments, the AR processing module 845 is configured process signals based at least on sensor data. In some embodiments, the AR processing module 845 is configured process signals based on image data received that captures at least a portion of the user hand, mouth, facial expression, surrounding, etc. For example, the housing 806 can receive EMG data and/or IMU data from one or more sensors 825 and provide the sensor data to the AR processing module 845 for a particular operation (e.g., gesture recognition, facial recognition, etc.). [0091] In some embodiments, the AR processing module 445 is configured to detect and determine one or more gestures performed by the user 115 based at least on sensor data. In some embodiments, the AR processing module 445 is configured detect and determine one or more gestures performed by the user 115 based on camera data received that captures at least a portion of the user 115's hand. For example, the wrist-wearable device 120 can receive EMG data and/or IMU data from one or more sensors 825 based on the user 115's performance of a hand gesture and provide the sensor data to the AR processing module 445 for gesture detection and identification. The AR processing module 445, based on the detection and determination of a gesture, causes a device communicatively coupled to the wrist-wearable device 120 to perform an operation (or action). In some embodiments, the AR processing module 445 is configured to receive sensor data and determine whether an image-capture trigger condition is satisfied. The AR processing module 845, causes a device communicatively coupled to the housing 806 to perform an operation (or action). In some embodiments, the AR processing module 845 performs different operations based on the sensor data and/or performs one or more actions based on the sensor data.

In some embodiments, the one or more imaging devices 855 can include an ultra-wide camera, a wide camera, a telephoto camera, a depth-sensing cameras, or other types of cameras. In some embodiments, the one or more imaging devices 855 are used to capture image data and/or video data. The imaging devices 855 can be coupled to a portion of the housing 806. The captured image data can be processed and stored in memory and then presented to a user for viewing. The one or more imaging devices 855 can include one or more modes for capturing image data or video data. For example, these modes can include a high-dynamic range (HDR) image capture mode, a low light image capture mode, burst image capture mode, and other modes. In some embodiments, a particular mode is automatically selected based on the environment (e.g., lighting, movement of the device, etc.). For example, a wrist-wearable device with HDR image capture mode and a low light image capture mode active can automatically select the appropriate mode based on the environment (e.g., dark lighting may result in the use of low light image capture mode instead of HDR image capture mode). In some embodiments, the user can select the mode. The image data and/or video data captured by the one or more imaging devices 855 is stored in memory 860 (which can include volatile and non-volatile memory such that the image data and/or video data can be temporarily or permanently stored, as needed depending on the circumstances).

The circuitry 846 is configured to facilitate the interaction between the housing 806 and the HMD 814. In some embodiments, the circuitry 846 is configured to regulate the distribution of power between the power source 807 and the HMD 814. In some embodiments, the circuitry 746 is configured to transfer audio and/or video data between the HMD 814 and/or one or more components of the housing 806.

The one or more processors 850 can be implemented as any kind of computing device, such as an integrated system-on-a-chip, a microcontroller, a fixed programmable gate array (FPGA), a microprocessor, and/or other application specific integrated circuits (ASICs). The processor may operate in conjunction with memory 860. The memory 860 may be or include random access memory (RAM), read-only memory (ROM), dynamic random access memory (DRAM), static random access memory (SRAM) and magnetoresistive random access memory (MRAM), and may include firmware, such as static data or fixed instructions, basic input/output system (BIOS), system functions, configuration data, and other routines used during the operation of the housing and the processor 850. The memory 860 also provides a storage area for data and instructions associated with applications and data handled by the processor 850.

In some embodiments, the memory 860 stores at least user data 861 including sensor data 862 and AR processing data 864. The sensor data 862 includes sensor data monitored by one or more sensors 825 of the housing 806 and/or sensor data received from one or more devices communicative coupled with the housing 806, such as the HMD 814, the smartphone 874b, the controller 874c, etc. The sensor data 862 can include sensor data collected over a predetermined period of time that can be used by the AR processing module 845. The AR processing data 864 can include one or more one or more predefined camera-control gestures, user defined camera-control gestures, predefined non-camera-control gestures, and/or user defined non-camera-control gestures. In some embodiments, the AR processing data 864 further includes one or more predetermined threshold for different gestures.

Further embodiments also include various subsets of the above embodiments including embodiments described with reference to FIGS. 1A-5 combined or otherwise re-arranged.

### Example Aspects

A few example aspects will now be briefly described.

(A1) In accordance with some embodiments, a method of using sensor data from a wrist-wearable device to monitor image-capture trigger conditions for determining when to capture images using an imaging device of a head-wearable device is disclosed. The head-wearable device and wrist-wearable device are worn by a user. The method includes receiving, from a wrist-wearable device communicatively coupled to a head-wearable device, sensor data; and determining, based on the sensor data received from the wrist-wearable device and without receiving an instruction from the user to capture an image, whether an image-capture trigger condition for the head-wearable device is satisfied. The method further includes, in accordance with a determination that the image-capture trigger condition for the head-wearable device is satisfied, instructing an imaging device of the head-wearable device to capture image data.

(A2) In some embodiments of A1, the sensor data received from the wrist-wearable device is from a first type of sensor, and the head-wearable device does not include the first type of sensor.

(A3) In some embodiments of any of A1 and A2, the method further includes receiving, from the wrist-wearable device that is communicatively coupled to the head-wearable device, additional sensor data; and determining, based on the additional sensor data received from the wrist-wearable device, whether an additional image-capture trigger condition for the head-wearable device is satisfied, the additional image-capture trigger condition being distinct from the image-capture trigger condition. The method further includes in accordance with a determination that the additional image-capture trigger condition for the head-wearable device is satisfied, instructing the imaging device of the head-wearable device to capture additional image data.

(A4) In some embodiments of A3, the method further includes, in accordance with the determination that the image-capture trigger condition for the head-wearable device is satisfied, instructing an imaging device of the wrist-wearable device to capture another image; and in accordance with the determination that the additional image-capture trigger condition for the head-wearable device is satisfied, forgoing instructing the imaging device of the wrist-wearable device to capture image data.

(A5) In some embodiments of A4, the method further includes in conjunction with instructing the imaging device of the wrist-wearable device to capture the other image, notifying the user to position the wrist-wearable device such that it is oriented towards a face of the user.

(A6) In some embodiments of A5, the imaging device of the wrist-wearable device is instructed to capture the other image substantially simultaneously with the imaging device of the head-wearable device capturing the image data.

(A7) In some embodiments of any of A1-A6, the determination that the image-capture trigger condition is satisfied is further based on sensor data from one or more sensors of the head-wearable device.

(A8) In some embodiments of any of A1-A7, the determination that the image-capture trigger condition is satisfied is further based on identifying, using data from one or both of the imaging device of the head-wearable device or an imaging device of the wrist-wearable device, a predefined object within a field of view of the user.

(A9) In some embodiments of any of A1-A8, the method further includes in accordance with the determination that the image-capture trigger condition is satisfied, instructing the wrist-wearable device to store information concerning the user's performance of an activity for association with the image data captured using the imaging device of the head-wearable device.

(A10) In some embodiments of any of A1-A9, the image-capture trigger condition is determined to be satisfied based on one or more of a target heartrate detected using the sensor data of the wrist-wearable device, a target distance during an exercise activity being monitored in part with the sensor data, a target velocity during an exercise activity being monitored in part with the sensor data, a target duration, a user-defined location detected using the sensor data, a user-defined elapsed time monitored in part with the sensor data, image recognition performed on image data included in the sensor data, and position of the wrist-wearable device and/or the head-wearable device detected in part using the sensor data.

(A11) In some embodiments of any of A1-A10, the instructing the imaging device of the head-wearable device to capture the image data includes instructing the imaging device of the head-wearable device to capture a plurality of images.

(A12) In some embodiments of any of A1-A11, the method further includes, after instructing the imaging device of the head-wearable device to capture the image data, in accordance with a determination that the image data should be shared with one or more other users, causing the image data to be sent to respective devices associated with the one or more other users.

(A13) In some embodiments of A12, the method further includes before causing the image data to be sent to the respective devices associated with the one or more other users, applying one or more of an overlay (e.g., can apply a hear rate to the captured image data, a running or completion time, a duration, etc.), a time stamp (e.g., when the image data was captured), geolocation data (e.g., where the image data was captured), and a tag (e.g., a recognized location or person that the user is with) to the image data to produce a modified image data that is then caused to be sent to the respective devices associated with the one or more other users.

(A14) In some embodiments of any of A12-A13, the method further includes before causing the image data to be sent to the respective devices associated with the one or more other users, causing the image data to be sent for display at the wrist-wearable device within an image-selection user interface. The determination that the image data should be shared with the one or more other users is based on a selection of the image data from within the image-selection user interface displayed at the wrist-wearable device.

(A15) In some embodiments of A14, the method further includes after the image data is caused to be sent for display at the wrist-wearable device, the image data is stored at the wrist-wearable device and is not stored at the head-wearable device.

(A16) In some embodiments of any of A12-A15, the determination that the image data should be shared with one or more other users is made when it is determined that the user has decreased their performance during an exercise activity.

(A17) In some embodiments of any of A1-A16, the method includes, in accordance with a determination that image-transfer criteria are satisfied, providing the captured image data to the wrist-wearable device.

(A18) In some embodiments of A17, the image-transfer criteria are determined to be satisfied due in part to the user of the wrist-wearable device completing or pausing an exercise activity.

(A19) In some embodiments of any of A1-A18, the method further includes receiving a gesture that corresponds to a handwritten symbol on a display of the wrist-wearable device and, responsive to the handwritten symbol, updating the display of the head-wearable device to present the handwritten symbol.

(B1) In accordance with some embodiments, a wrist-wearable device configured to use sensor data to monitor image-capture trigger conditions for determining when to capture images using a communicatively coupled imaging device is provided. The wrist-wearable device includes a display, one or more sensors, and one or more processors. The communicatively coupled imaging device can be coupled with a head-wearable device. The head-wearable device and wrist-wearable device are worn by a user. The one or more processors are configured to receive, from the one or more sensors, sensor data; and determine, based on the sensor data and without receiving an instruction from the user to capture an image, whether an image-capture trigger condition for the head-wearable device is satisfied. The one or more processors are further configured to in accordance with a determination that the image-capture trigger condition for the head-wearable device is satisfied, instruct an imaging device of the head-wearable device to capture image data.

(B2) In some embodiments of B1, the wrist-wearable device is further configured to perform operations of the wrist-wearable device recited in the method of any of A2-A19.

(C1) In accordance with some embodiments, a head-wearable device configured to use sensor data from a wrist-wearable device to monitor image-capture trigger conditions for determining when to capture images using an communicatively coupled imaging device is provided. The head-wearable device and wrist-wearable device are worn by a user. The head-wearable device includes a heads-up display, an imaging device, one or more sensors, and one or more processors. The one or more processors are configured to receive, from a wrist-wearable device communicatively coupled to a head-wearable device, sensor data; and determine, based on the sensor data received from the wrist-wearable device and without receiving an instruction from the user to capture an image, whether an image-capture trigger condition for the head-wearable device is satisfied. The one or more processors are further configured to in accordance with a determination that the image-capture trigger condition for the head-wearable device is satisfied, instruct the imaging device to capture an image data.

(C2) In some embodiments of C1, the head-wearable device is further configured to perform operations of the head-wearable device recited in the method of any of A2-A19.

(D1) In accordance with some embodiments, a system for using sensor data to monitor image-capture trigger conditions for determining when to capture images using a communicatively coupled imaging device is provided. The system includes a wrist-wearable device and a head-wearable device. The head-wearable device and wrist-wearable device are worn by a user. The wrist-wearable device includes a display, one or more sensors, and one or more processors. The one or more processors of the wrist-wearable device are configured to at least monitor sensor data while worn by the user. The head-wearable device includes a heads-up display, an imaging device, one or more sensors, and one or more processors. The one or more processors of the head-wearable device are configured to at least monitor sensor data while worn by the user. The system is configured to receive, from a wrist-wearable device communicatively coupled to a head-wearable device, sensor data; and determine, based on the sensor data received from the wrist-wearable device and without receiving an instruction from the user to capture an image, whether an image-capture trigger condition for the head-wearable device is satisfied. The system is further configured to in accordance with a determination that the image-capture trigger condition for the head-wearable device is satisfied, instruct the imaging device to capture an image data.

(D2) In some embodiments of D1, the system is further configured such that the wrist-wearable device performs operations of the wrist-wearable device recited in the method of any of claims 2-18 and the head-wearable device performs operations of the head-wearable device recited in the method of any of claims 2-19.

(E1) In accordance with some embodiments, a wrist-wearable device including means for causing performance of any of A1-A19.

(F1) In accordance with some embodiments, a head-wearable device including means for causing performance of any of A1-A19.

(G1) In accordance with some embodiments, an intermediary device configured to coordinate operations of a wrist-wearable device and a head-wearable device, the intermediary device configured to perform or cause performance of any of A1-A19.

(H1) In accordance with some embodiments, non-transitory, computer-readable storage medium including instructions that, when executed by a head-wearable device, a wrist-wearable device, and/or an intermediary device in communication with the head-wearable device and/or the wrist-wearable device, cause performance of the method of any of A1-A19.

(I1) In accordance with some embodiments, a method including receiving sensor data from a wrist-wearable device worn by a user indicating performance of an in-air hand gesture associated with unlocking access to a physical item, and in response to receiving the sensor data, causing an imaging device of a head-wearable device that is communicatively coupled with the wrist-wearable device to capture image data. The method further includes, in accordance with a determination that an area of interest in the image data satisfies an image-data-searching criteria, identifying a visual identifier within the area of interest in the image data, and after determining that the visual identifier within the area of interest in the image data is associated with unlocking access to the physical item, providing information to unlock access to the physical item.

(I2) In some embodiments of I1, the method further includes before the determination that the area of interest in the image data satisfies the image-data-searching criteria is made, presenting of the area of interest in the image data at the head-wearable device as zoomed-in image data.

(I3) In some embodiments of I2, the visual identifier is identified within the zoomed-in image data.

(I4) In some embodiments of any of I1-I3, the area of interest in the image data is presented with an alignment marker, and the image-data-searching criteria is determined to be satisfied when it is determined that the visual identifier is positioned with respect to the alignment marker.

(I5) In some embodiments of any of I1-I4, the determination that the area of interest in the image data satisfies the image-data-searching criteria is made is in response to a determination that the head-wearable device is positioned in a stable downward position.

(I6) In some embodiments of any of I1-I5, the visual identifier includes one or more of a QR code, a barcode, a writing, a label, and an object identified by an image-recognition algorithm.

(I7) In some embodiments of any of I1-I6, the physical item is a bicycle available for renting.

(I8) In some embodiments of any of I1-I7, the physical item is a locked door.

(I9) In some embodiments of any of I1-I8, the method further includes, before identifying the visual identifier, and in accordance with a determination that an additional area of interest in the image data fails to satisfy the image-data searching criteria, forgoing identifying a visual identifier within the additional area of interest in the image data.

(I10) In some embodiments of any of I1-I9, the method further includes, before determining that the visual identifier within the area of interest in the image data is associated with unlocking access to the physical item, and in accordance with a determination that the visual identifier is not associated with unlocking access to the physical item, forgoing providing information to unlock access to the physical item.

(I11) In some embodiments of any of I1-I10, the method further includes causing the imaging device of the head-wearable device that is communicatively coupled with the wrist-wearable device to capture second image data in response to receiving a second sensor data. The method also further includes, in accordance with a determination that a second area of interest in the second image data satisfies a second image-data-searching criteria, identifying a second visual identifier within the second area of interest in the second image data. The method also further includes, after determining that the second visual identifier within the second area of interest in the second image data is associated with unlocking access to a second physical item, providing second information to unlock access to the second physical item.

(J1) In accordance with some embodiments, a head-wearable device for adjusting a representation of a user's position within an artificial-reality application using a hand gesture, the head-wearable device configured to perform or cause performance of the method of any of I1-I11.

(K1) In accordance with some embodiments, a system for adjusting a representation of a user's position within an artificial-reality application using a hand gesture, the system configured to perform or cause performance of the method of any of I1-I11.

(L1) In accordance with some embodiments, non-transitory, computer-readable storage medium including instructions that, when executed by a head-wearable device, a wrist-wearable device, and/or an intermediary device in communication with the head-wearable device and/or the wrist-wearable device, cause performance of the method of any of I1-I11.

(M1) In another aspect, a means on a wrist-wearable device, head-wearable device, and/or intermediary device for performing or causing performance of the method of any of I1-I11.

Any data collection performed by the devices described herein and/or any devices configured to perform or cause the performance of the different embodiments described above in reference to any of the Figures, hereinafter the “devices,” is done with user consent and in a manner that is consistent with all applicable privacy laws. Users are given options to allow the devices to collect data, as well as the option to limit or deny collection of data by the devices. A user is able to opt-in or opt-out of any data collection at any time. Further, users are given the option to request the removal of any collected data.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

What is claimed is:

1. A method of using sensor data from a wrist-wearable device to monitor image-capture trigger conditions for determining when to capture images using an imaging device of a head-wearable device, the method comprising:
receiving, from a wrist-wearable device communicatively coupled to a head-wearable device, sensor data, wherein the head-wearable device and wrist-wearable device are worn by a user;
determining, based on the sensor data received from the wrist-wearable device and without receiving an instruction from the user to capture an image, whether an image-capture trigger condition for the head-wearable device is satisfied; and
in accordance with a determination that the image-capture trigger condition for the head-wearable device is satisfied, instructing an imaging device of the head-wearable device to capture image data.

2. The method of claim 1, wherein:
the sensor data received from the wrist-wearable device is from a first type of sensor, and
the head-wearable device does not include the first type of sensor.

3. The method of claim 1, further comprising:
receiving, from the wrist-wearable device that is communicatively coupled to the head-wearable device, additional sensor data;
determining, based on the additional sensor data received from the wrist-wearable device, whether an additional image-capture trigger condition for the head-wearable device is satisfied, the additional image-capture trigger condition being distinct from the image-capture trigger condition; and
in accordance with a determination that the additional image-capture trigger condition for the head-wearable device is satisfied, instructing the imaging device of the head-wearable device to capture additional image data.

4. The method of claim 3, further comprising:
in accordance with the determination that the image-capture trigger condition for the head-wearable device is satisfied, instructing an imaging device of the wrist-wearable device to capture another image; and
in accordance with the determination that the additional image-capture trigger condition for the head-wearable device is satisfied, forgoing instructing the imaging device of the wrist-wearable device to capture image data.

5. The method of claim 4, further comprising:
in conjunction with instructing the imaging device of the wrist-wearable device to capture the other image, notifying the user to position the wrist-wearable device such that it is oriented towards a face of the user.

6. The method of claim 1, wherein the determination that the image-capture trigger condition is satisfied is further based on sensor data from one or more sensors of the head-wearable device.

7. The method of claim 1, wherein the determination that the image-capture trigger condition is satisfied is further based on identifying, using data from one or both of the imaging device of the head-wearable device or an imaging device of the wrist-wearable device, a predefined object within a field of view of the user.

8. The method of claim 5, wherein:
the imaging device of the wrist-wearable device is instructed to capture the other image substantially simultaneously with the imaging device of the head-wearable device capturing the image data.

9. The method of claim 1, further comprising:
in accordance with the determination that the image-capture trigger condition is satisfied, instructing the wrist-wearable device to store information concerning the user's performance of an activity for association with the image data captured using the imaging device of the head-wearable device.

10. The method of claim 1, wherein the image-capture trigger condition is determined to be satisfied based on one or more of a target heartrate detected using the sensor data of the wrist-wearable device, a target distance during an exercise activity being monitored in part with the sensor data, a target velocity during an exercise activity being monitored in part with the sensor data, a target duration, a user-defined location detected using the sensor data, a user-defined elapsed time monitored in part with the sensor data, image recognition performed on image data included in the sensor data, and position of the wrist-wearable device and/or the head-wearable device detected in part using the sensor data.

11. The method of claim 1, wherein instructing the imaging device of the head-wearable device to capture the image data includes instructing the imaging device of the head-wearable device to capture a plurality of images.

12. The method of claim 1, further comprising:
after instructing the imaging device of the head-wearable device to capture the image data:
in accordance with a determination that the image data should be shared with one or more other users, causing the image data to be sent to respective devices associated with the one or more other users.

13. The method of claim 12, further comprising:
before causing the image data to be sent to the respective devices associated with the one or more other users, applying one or more of an overlay, a time stamp, geolocation data, and a tag to the image data to produce a modified image data that is then caused to be sent to the respective devices associated with the one or more other users.

14. The method of claim 12, further comprising:
before causing the image data to be sent to the respective devices associated with the one or more other users, causing the image data to be sent for display at the wrist-wearable device within an image-selection user interface,
wherein the determination that the image data should be shared with the one or more other users is based on a selection of the image data from within the image-selection user interface displayed at the wrist-wearable device.

15. The method of claim 14, further comprising:
after the image data is caused to be sent for display at the wrist-wearable device, the image data is stored at the wrist-wearable device and is not stored at the head-wearable device.

16. The method of claim 12, wherein the determination that the image data should be shared with one or more other users is made when it is determined that the user has decreased their performance during an exercise activity.

17. The method of claim 1, further comprising:
receiving a gesture that corresponds to a handwritten symbol on a display of the wrist-wearable device; and
responsive to the handwritten symbol, updating the display of the head-wearable device to present the handwritten symbol.

18. The method of claim 1, the method further comprising:
in accordance with a determination that an area of interest in the image data satisfies an image-data-searching criteria, identifying a visual identifier within the area of interest in the image data; and
after determining that the visual identifier within the area of interest in the image data is associated with unlocking access to a physical item, providing information to unlock access to the physical item.

19. A wrist-wearable device configured to use sensor data to monitor image-capture trigger conditions for determining when to capture images using a communicatively coupled imaging device, the wrist-wearable device comprising:
a display;
one or more sensors; and
one or more processors configured to:
receive, from the one or more sensors, sensor data;
determine, based on the sensor data, whether an image-capture trigger condition for a communicatively coupled head-wearable device is satisfied; and
in accordance with a determination that the image-capture trigger condition for the communicatively coupled head-wearable device is satisfied, instruct an imaging device of the communicatively coupled head-wearable device to capture image data.

20. A non-transitory, computer-readable storage medium including instructions that, when executed by a wrist-wearable device, cause the wrist-wearable device to:
receive, via one or more sensors communicatively coupled with the wrist-wearable device, sensor data;
determine, based on the sensor data, whether an image-capture trigger condition for a communicatively coupled head-wearable device is satisfied; and
in accordance with a determination that the image-capture trigger condition for the head-wearable device is satisfied, instruct an imaging device of the head-wearable device to capture image data.
